(19) United States
(12) Patent Application Publication
(10) Pub. No.: US 2023/0338554 A1
(43) Pub. Date: Oct. 26, 2023

(54) GALACTOSYLATED DENDRIMERS FOR TARGETED INTRACELLULAR DELIVERY TO HEPATOCYTES

(71) Applicant: The Johns Hopkins University, Baltimore, MD (US)

(72) Inventors: Kannan Rangaramanujam, Highland, MD (US); Rishi Sharma, Baltimore, MD (US); Anjali Sharma, Baltimore, MD (US); Joshua Porterfield, Towson, MD (US); Sujatha Kannan, Highland, MD (US)

(21) Appl. No.: 18/044,405

(22) PCT Filed: Sep. 8, 2021
(86) PCT No.: PCT/US2021/049392

§ 371 Date: Mar. 8, 2023

Related U.S. Application Data
(60) Provisional application No. 63/075,499, filed on Sep. 8, 2020.

(51) Int. Cl. A61K 31/355; C08G 83/00; A61K 45/06; A61K 47/59

(52) U.S. Cl. CPC A61K 47/595; A61K 31/355; A61K 45/06; C08G 83/003

(57) ABSTRACT

Compositions of dendrimers conjugated with galactose and one or more active agents to prevent, treat or diagnose a liver injury, liver disease or liver disorder in a subject in need thereof, and methods of use thereof, have been developed. Preferably, the therapeutic agents are one or more anti-inflammatory agents. The compositions are particularly suited for treating and/or ameliorating one or more symptoms of non-alcoholic steatohepatitis and severe acetaminophen poisoning. Methods of treating a human subject having or at risk of non-alcoholic steatohepatitis and severe acetaminophen poisoning are provided.

### CROSS REFERENCE TO RELATED APPLICATION

This application claims benefit of U.S. Provisional Application No. 63/075,499, filed Sep. 8, 2020 which, is hereby incorporated herein by reference in its entirety.

### STATEMENT REGARDING FEDERALLY SPONSORED RESEARCH

This invention was made with government support under EY001765 and HD076901 awarded by the National Institutes of Health. The government has certain rights in the invention.

### FIELD OF THE INVENTION

The invention is generally in the field of drug delivery, and in particular, methods for delivering drugs selectively to hepatocytes via dendrimer formulations.

### BACKGROUND OF THE INVENTION

The liver is the second largest and one of the most vital organs of the human body, which remarkably performs a plethora of metabolic, immunological and endocrine functions including the storage of glycogen, secretion of bile for digestion, detoxification of blood, breakdown of fat, proteins, and carbohydrates, and maintenance of homeostasis by elimination of exogenous and endogenous compounds. A perturbation or disruption in the regulatory mechanisms of liver metabolism can lead to liver dysfunction, causing severe systemic complications. Recently, incidences of liver diseases have been increasing in prevalence with more than 844 million people around the globe suffering from a chronic liver problem, and around 2 million people every year dying from liver disorders worldwide. In North America, there has been a 10-15% increase in the rate of liver transplant in recent years, which is an enormous stress on an already overburdened system. Despite these facts, the current treatment options for most of these hepatic diseases are limited and lack the necessary efficacy in advanced and severe cases. Hepatocytes are the most abundant liver cell type, constituting more than 80% of the liver biomass and are predominantly implicated in most liver disorders such as hepatocellular carcinoma, drug induced liver failure, hepatitis, and non-alcoholic steatohepatitis. Although many drugs and nanoparticles administered systemically accumulate in the liver due to the reticuloendothelial system, their delivery to hepatocytes is hampered by macrophages and Kupffer cells, which rapidly sequester drugs before reaching hepatocytes in the desired therapeutic concentrations. Even the drugs that do end up in hepatocytes can quickly be removed without having a pharmacological effect, due to hepatobiliary clearance.

The lack of effective site-specific delivery of drugs to hepatocytes, and their off-target interactions, are the primary challenges to treatment of liver diseases. Achieving this goal would increase the bioavailability of drugs, as well as reduce their dose-related side effects to treat hepatic malignancies, decrease the need for liver transplants and increase life expectancy. Despite abundant reports of liver targeting nanoparticles, translation of hepatocyte-targeting nanotherapeutics has not seen clinical success so far. In addition to the challenging hepatocyte-specific targeting properties, other hurdles in the clinical translation and commercialization of hepatocyte-targeting nanoparticles relate to their cytotoxicity, scale-up, structural defects, synthetic reproducibility, product purity, in vivo stability, and short half-life.

Therefore, it is an object of the invention to provide compositions that selectively target hepatocytes, and methods of making and using thereof.

It is also an object of the invention to provide compositions for the treatment or prevention of one or more symptoms of liver diseases and/or disorders, in particular, non-alcoholic steatohepatitis and severe acetaminophen poisoning.

It is yet another object of the invention to provide compositions and methods for selectively targeting active agents to hepatocytes, and methods of making and using thereof.

### SUMMARY OF THE INVENTION

Conjugation of dendrimer molecules with galactose carbohydrate moieties drives selective uptake of the dendrimers by hepatocyte cells in vivo. Compositions and methods of galactosylated dendrimers complexed to, covalently conjugated to, or having intra-molecularly dispersed or encapsulated therein one or more therapeutic or prophylactic agents for treating or preventing liver diseases are described.

Methods for treating or preventing one or more symptoms of a liver disease and/or disorder in a subject in need thereof include administering to the subject a formulation of galactosylated dendrimers having bound or complexed thereto one or more therapeutic or prophylactic agents. The formulation is delivered to the subject in an amount effective to treat, alleviate or prevent one or more symptoms of a liver disease and/or disorder. Exemplary liver diseases and disorders that can be treated or prevented include inflammatory liver diseases, non-alcoholic steatohepatitis, drug-induced liver failure, hepatitis, liver fibrosis, and liver cirrhosis. Exemplary therapeutic agents complexed or conjugated with the galactosylated dendrimers include non-steroidal anti-inflammatory agents, corticosteroid anti-inflammatory agents, gold compound anti-inflammatory agents, immunosuppressive, and anti-oxidant agents. An exemplary anti-inflammatory agent is N-acetyl cysteine. In some forms, the therapeutic agent is vitamin E.

In some forms, the galactosylated dendrimers within the formulation are poly(amidoamine) (PAMAM) dendrimers, for example, generation 4, generation 5, generation 6, generation 7, or generation 8 PAMAM dendrimers. Exemplary galactosylated dendrimers include hydroxyl-terminated dendrimers. In some forms, galactosylated, hydroxyl-terminated dendrimers are formed of galactose and oligoethylene glycol building blocks. Exemplary galactosylated, hydroxyl-terminated dendrimers are formed of galactose and oligoethylene glycol building blocks having 24 hydroxyl terminal groups (generation 1), 96 hydroxyl terminal groups (generation 2), 384 hydroxyl terminal groups (generation 3), or 1,536 hydroxyl terminal groups (generation 4). In some forms, the galactosylated, hydroxyl-terminated dendrimers include an outer layer of galactose moieties and a dendrimer backbone. In some forms, the dendrimer backbone also includes galactose moieties embedded therein. An exemplary galactosylated, hydroxyl-terminated generation 2 dendrimer, includes 24 galactose moieties forming the outer layer and six galactose moieties embedded in the dendrimer backbone. Galactose moieties embedded in the dendrimer backbone are typically connected through tetraethylene glycol units.

In some forms, formulations of galactosylated, hydroxyl-terminated dendrimers are administered in an amount effective to reduce the serum levels of one or more biomarkers in the recipient. Exemplary biomarkers include alanine aminotransferase (ALT), aspartate aminotransferase (AST), triglyceride (TG), gamma-glutamyltrasferase (GGT), total cholesterol (TC), low-density lipoprotein (LDP), and fasting blood sugar. In other forms, the methods administer formulations of galactosylated, hydroxyl-terminated dendrimers in an amount effective to induce one or more pathophysiological changes in the recipient. For example, in some forms, the methods reduce one or more of steatosis, inflammation, ballooning, fibrosis, or cirrhosis, in the subject. For example, in some forms, the methods administer formulations in an amount effective to reduce lobular inflammation in the liver; to reduce the amount or presence of one or more pro-inflammatory cells, chemokines, and/or cytokines in the liver; or to reduce one or more pro-inflammatory cytokines in the recipient. Typically, galactosylated, hydroxyl-terminated dendrimers are formulated for systemic administration, for example, for administration via intravenous or intraperitoneal administration, or via oral administration.

In some forms, the methods administer formulations of galactosylated, hydroxyl-terminated dendrimers prior to, in conjunction with, subsequent to, or in alternation with treatment with one or more additional therapies or procedures. Exemplary additional procedures include administering one or more therapeutic, prophylactic and/or diagnostic agents to prevent or treat one or more symptoms of associated diseases or conditions of liver injuries, such as infections, sepsis, diabetic complications, hypertension, obesity, high blood pressure, heart failure, kidney diseases, and cancers.

Pharmaceutical formulations of galactosylated, hydroxyl-terminated dendrimers complexed or conjugated with one or more therapeutic, prophylactic or diagnostic agents are also described.

Methods of making dendrimers with a plurality of surface galactose moieties are also described. Typically, the methods include the steps of (a) preparing a hypercore by propargylation of a first monomer, wherein the first monomer includes two or more reactive groups for propargylation; (b) conjugating one azide group onto glycosidic linkage at C1 of galactose via a orthogonal polyalkylene oxide linker, preferably a PEG4 linker, to generate a galactose hyper monomer; (c) mixing the hypercore and the galactose hyper monomer for copper (I) catalyzed alkyne azide click chemistry to yield a generation 1 dendrimer; (d) conjugating allyl groups on four of the reactive groups of the galactose hyper monomer on the generation 1 dendrimer; and (e) mixing the generation 1 dendrimer with β-Gal-PEG4-azide for copper (I) catalyzed alkyne azide click chemistry to yield generation 2 dendrimers. The same process can be used to make additional generation polymers. In an exemplary method, the hypercore in step (a) is a hexa-propargylated core. In a further exemplary method, the generation 2 dendrimer from step (e) is a dendrimer with 24 galactose units forming an outer layer, and six galactose units embedded within the dendrimer backbone. The six galactose units embedded in the core are connected through tetraethylene glycol units. Methods of complexing and/or conjugating dendrimers with a plurality of surface galactose moieties to one or more therapeutic, prophylactic, and/or diagnostic agents are also described.

### BRIEF DESCRIPTION OF THE DRAWINGS

FIGS. 1A and 1B are schematics showing the step-wise synthetic route for producing generation 2 Galactose-24 dendrimer (GAL-24, FIG. 1A); and fluorescent labeling of GAL-24 (GAL24-Cy5, FIG. 1B). Synthesis in FIG. 1A starts from a hexapropargylated core (1); with addition of AB4 building blocks (2) by CuAAC click reaction; forming G1-Galactose-6; and G1-Galactose-6-propargyl 24 (4); and subsequent extension to G2 Galactose 24-OH (5b) via G2 Galactose 24-OAc intermediate (5A). Labelling of GAL-24 in FIG. 1B with Cy5 to obtain GAL24-Cy5 is achieved via a two-step reaction procedure. Conditions: (i) CuSO4.5H2O, sodium ascorbate, THF, H₂O, DMF, 50° C., 8h, microwave; (ii) sodium methoxide, methanol, pH 8.5-9.0, room temperature, overnight; (iii) NaH, propargyl bromide 0° C. to room temperature, 8h.

FIG. 2A is a bar graph showing % cell viability of three immortalized human cell lines, HEPG2 (hepatocarcinoma); HMC3 (macrophages); and HUVEC (endothelial cells) at a concentration of 0, 0.01, 0.1,1, 10, 100 and 1,000 µg/mL of GAL-24 (n=3), respectively. FIG. 2B is a line graph showing concentration of free GAL-24 dendrimer (nM) versus concentrations of bound dendrimers (nM) using cellular binding assay involving HEPG2 cells. FIG. 2C is a bar graph showing uptake of GAL24-Cy5 by HEPG2 cells (pg/cell) when incubated with 20 µg/mL GAL-24-Cy5 only (n=8), or co-incubation with free galactose (20 mM, n=6), or chlorpromazine (10 µg/mL, n=7), respectively.

FIGS. 3A and 3B are bar graphs showing GAL-24 liver localization in healthy C57BL6 mice. FIG. 3A shows the percentage of injected GAL-24 in liver at 1, 4, 24, and 48 hours post tail vein administration of GAL-24. FIG. 3B is a bar graph showing percentages of cells containing GAL24-Cy5 in hepatocytes (ASGP-R positive) and non-hepatocytes (ASGP-R negative) populations 24 hours after injected with GAL-24-Cy5.

FIG. 4 is a bar graph showing the percentage of injected GAL-24 in each of brain, spleen, heart, lungs, and kidney tissues, as well as plasma at 1, 4, 24, and 48 hours post tail vein administration of GAL24-Cy5, respectively.

FIG. 5 is a bar graph showing Gal24-Cy5 concentrations (µg/g tissue) in liver tissue in mice administered with or without an overdose (700 mg/kg) of acetaminophen.

FIG. 6 is a schematic showing synthesis of targeted dendrimer G4-Galctose-Cy5 (GAL-D4-Cy5; 10) having galactose as targeting ligands and Cy5 as an imaging agent from generation 4 hydroxyl-terminated PAMAM dendrimer (PAMAM-G4-OH, 1).

FIGS. 7A-7C are graphs showing assessment of D4-GAL asialoglycoprotein receptor (ASGPR) binding and hepatocellular uptake in vitro. FIG. 7A is a line graph showing concentrations (nM) of D4-OH dendrimer bound to ASGPR in vitro at various concentrations (nM) of free D4-OH dendrimer. FIG. 7B is a line graph showing concentrations (nM) of D4-GAL dendrimer bound to ASGPR in vitro at various concentrations (nM) of free D4-GAL dendrimer. FIG. 7C is a bar graph showing dendrimer uptake (pg/cell) of D4-Cy5 or Gal-D4-Cy5 with or without 20 mM free galactose.

FIGS. 8A and 8B are bar graphs showing liver and hepatocyte localization of Gal-D4-Cy5 upon systemic administration. FIG. 8A shows percentage of injected D4-Cy5 or GAL-D4-Cy5 in liver at 1, 4, 24, and 48 hours after tail vein administration, respectively. FIG. 8B is a bar graph showing percentages of cells containing D4-Cy5 or GAL-D4-Cy5 in hepatocytes (ASGP-R positive) and non-hepatocytes (ASGP-R negative) populations 24 hours after injected with GAL-24-Cy5.

FIGS. 9A and 9B are bar graphs showing pharmacokinetics and biodistribution of D4-Cy5 or GAL-D4-Cy5. FIG. 9A is a bar graph showing percentage of injected D4-Cy5 or GAL-D4-Cy5 in each of brain, spleen, heart, lung, and kidney tissues, respectively, at 1, 4, 24, and 48 hours post tail vein administration of D4-Cy5 or GAL-D4-Cy5. FIG. 9B is a bar graph showing percentage of injected D4-Cy5 or GAL-D4-Cy5 in serum at 1, 4, 24, and 48 hours post tail vein administration of D4-Cy5 or GAL-D4-Cy5.

FIG. 10 is a bar graph showing GAL-D4-Cy5 or D4-Cy5 uptake in the liver (%ID/g tissue) of each of healthy mice; mice 24 hours following an overdose of acetaminophen (APAP); and rats fed a high fat methionine and choline deficient diet for 6 weeks to induce non-alcoholic steatohepatitis (NASH), respectively.

FIG. 11 is a schematic showing synthesis of G4-GalactoseNAC (Gal-D4-NAC; 16) having galactose as targeting ligands and NAC as therapeutic agent.

FIG. 12A is a diagram showing experimental set up and disease pathogenesis; on day 1 initial mass is recorded and food restriction begins; on day 1 after 4 hrs, Acetaminophen is formulated at 25 mg/ml in 10% DMSO in saline and injected at 700 mg/kg (i.p.) based on initial mass; on day 2 food is returned after acetaminophen absorption has begun; five and a half hours later, Gal-D4-NAC in saline, free NAC in saline or saline is administered (i.v.); mass is monitored until 80% of initial mass is reached, then animal is sacrificed; or animals are sacrificed 24 hrs after Gal-D4-NAC, with serum and liver collected for analysis. FIG. 12B is a graph showing percent survival of test animals treated with either nothing (sham); saline; free NAC, or Gal-D4-NAC, respectively, as determined by mass reduction below 80% of the initial animal mass, over time (hours) following an overdose of acetaminophen (APAP) .

### DETAILED DESCRIPTION OF THE INVENTION

### I. Definitions

The terms “active agent” or “biologically active agent” are used interchangeably to refer to a chemical or biological compound that induces a desired pharmacological and/or physiological effect, which may be prophylactic, therapeutic or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind hepatocytes. The term “prophylactic agent” generally refers to an agent that can be administered to prevent disease or to prevent certain conditions, such as a vaccine.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more liver diseases or disorders, such as inhibiting or reducing serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of nSMase2 associated activated microglia by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at mRNAs, proteins, cells, tissues and organs levels. For example, an inhibition and reduction in the rate of liver fat accumulation or steatosis, inflammation, ballooning, fibrosis, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with NASH disease are mitigated or eliminated, including, but are not limited to, reducing serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality.

The term “analog” refers to a chemical compound with a structure similar to that of another “reference” compound, but differing from it in respect to a particular component, functional group, atom, etc.

The term “derivative” refers to a compound, which is formed from a parent compound by one or more chemical reaction(s).

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The phrase “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The entity may be, for example, a therapeutic compound such as a small molecule, or a diagnostic entity such as a detectable label. The location may be a tissue, a particular cell type, or a subcellular compartment. In one embodiment, the targeting moiety directs the localization of an agent. In preferred embodiment, the dendrimer composition can selectively target activated microglia in the absence of an additional targeting moiety.

The term “prolonged residence time” refers to an increase in the time required for an agent to be cleared from a patient’s body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

### II. Compositions

It has been established that dendrimers (D) conjugated or complexed with galactose (Gal) selectively accumulate within hepatocyte cells. Compositions of dendrimers conjugated or complexed with galactose (D-Gal) suitable for delivering one or more agents, particularly one or more agents to prevent, treat, or diagnose one or more liver disorders and/or diseases, in a subject in need thereof, have been developed. The D-Gal compositions are particularly suited for treating and/or ameliorating one or more symptoms of non-alcoholic steatohepatitis and severe acetaminophen poisoning.

Compositions of D-Gal include one or more prophylactic, therapeutic, and/or diagnostic agents encapsulated, associated, and/or conjugated with the galactosylated dendrimers. Generally, one or more active agent is encapsulated, associated, and/or conjugated in the dendrimer complex at a concentration of about 0.01% to about 30% by weight, preferably about 1% to about 20%, more preferably about 5% to about 20% by weight. Preferably, an active agent is covalently conjugated to the dendrimer via one or more linkages such as disulfide, ester, ether, thioester, carbamate, carbonate, hydrazine, and amide, optionally via one or more spacers. In some embodiments, the spacer is an agent such as N-acetyl cysteine. Exemplary active agents include anti-inflammatory drugs and anti-oxidant agents.

The presence of active agents can affect the zeta-potential or the surface charge of the dendrimer-galactose complexes. In one embodiment, the zeta potential of the dendrimer-galactose complexes conjugated or complexed with active agent(s) is between -100 mV and 100 mV, between -50 mV and 50 mV, between -25 mV and 25 mV, between -20 mV and 20 mV, between -10 mV and 10 mV, between -10 mV and 5 mV, between -5 mV and 5 mV, or between -2 mV and 2 mV. The range above is inclusive of all values from -100 mV to 100 mV. In a preferred embodiment, the surface charge is neutral or near-neutral, i.e., from about -10 mV to about 10 mV, inclusive, preferably from about -1 mV to about 1 mV.

### A. Dendrimers

Dendrimers are three-dimensional, hyperbranched, monodispersed, globular and polyvalent macromolecules including a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nanocarriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Recent studies have shown that dendrimer surface groups have a significant impact on their biodistribution (Nance, E., et al., Biomaterials, 101, 96 (2016)). Hydroxyl terminated generation 4 PAMAM dendrimers (~4 nm size) without any targeting ligand cross the impaired blood/brain barrier (BBB) upon systemic administration in a rabbit model of cerebral palsy (CP) significantly more (> 20 fold) as compared to healthy controls, and selectively target activated microglia and astrocytes (Lesniak, W. G., et al., Mol Pharm, 10 (2013)).

The term “dendrimer” (“D”) includes, but is not limited to, a molecular architecture with an interior core and layers, or “generations” of repeating units which are attached to and extend from this interior core, each layer having one or more branching points, and an exterior surface of terminal groups attached to the outermost generation. In some embodiments, dendrimers have regular dendrimeric or “starburst” molecular structures. The dendrimers can have carboxylic, amine, or hydroxyl terminations, and can be of any generation including, but not limited to, generation 1 (“G1”) dendrimers (“D1”), generation 2 (“G2”) dendrimers (“D2”), generation 3 (“G3”) dendrimers (“D3”), generation 4 (“G4”) dendrimers (“D4”), generation 5 (“G5”) dendrimers (“D5”), generation 6 (“G6”) dendrimers (“D6”), generation 7 (“G7”) dendrimers (“D7”), generation 8 (“G8”) dendrimers (“D8”), generation 9 (“G9”) dendrimers (“D9”), or generation 10 (“G10”) dendrimers (“D10”).

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In general, active agents are encapsulated in a ratio of agent to dendrimer of between 1:1 and 4:1 for the larger generation dendrimers. In preferred embodiments, the dendrimers have a diameter effective to penetrate brain tissue and to retain in target cells for a prolonged period of time.

In some embodiments, dendrimers have a molecular weight between about 500 Daltons and about 100,000 Daltons, preferably between about 500 Daltons and about 50,000 Daltons, most preferably between about 1,000 Daltons and about 20,000 Daltons.

Suitable dendrimer scaffolds that can be used include poly(amidoamine), also known as PAMAM, or STARBURST™ dendrimers, polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. In preferred embodiments, the dendrimers have hydroxyl terminations. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 (G1) PAMAM dendrimers, generation 2 (G2) PAMAM dendrimers, generation 3 (G3) PAMAM dendrimers, generation 4 (G4) PAMAM dendrimers, generation 5 (G5) PAMAM dendrimers, generation 6 (G6) PAMAM dendrimers, generation 7 (G7) PAMAM dendrimers, generation 8 (G8) PAMAM dendrimers, generation 9 (G9)PAMAM dendrimers, or generation 10 (G10) PAMAM dendrimers. In the preferred embodiment, the dendrimers are soluble in the formulation and are generation (“G”) 4, 5 or 6 dendrimers (D4, D5, or D6). The dendrimers may have hydroxyl groups attached to their functional surface groups.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and active agents bound or complexed/conjugated thereto.

In some embodiments, the dendrimers include a plurality of hydroxyl groups. Some exemplary high-density hydroxyl group-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (“D2-OH-60”) can be synthesized using highly efficient, robust and atom economical chemical reactions such as Cu (I) catalyzed alkyne-azide click and photo catalyzed thiol-ene click chemistry. Highly dense polyol dendrimers of low generation in minimum reaction steps can be produced by using an orthogonal hypermonomer and hypercore strategy, for example as described in International Patent Publication No. WO 2019/094952. In some embodiments, the dendrimer backbone has non-cleavable polyether bonds throughout the structure to avoid disintegration of dendrimer in vivo and to allow the elimination of such non-biodegradable dendrimers as a single entity from the body.

In some embodiments, the dendrimer specifically targets a particular tissue region and/or cell type following administration into the body. In preferred embodiments, the dendrimer specifically targets a particular tissue region and/or cell type without a targeting moiety.

In preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50. In further embodiments, the surface density of hydroxyl (-OH) groups is between about 1 and about 50, preferably 5-20 OH group/nm² (number of hydroxyl surface groups/surface area in nm²) while having a molecular weight of between about 500 Da and about 10 kDa.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50. In some embodiments, the volumetric density of hydroxyl groups is between about 4 and about 50 groups/nm³, preferably between about 5 and about 30 groups/nm³, more preferably between about 10 and about 20 groups/nm³.

### I. Methods of Making Dendrimers

Dendrimers can be prepared via a variety of chemical reaction steps. Dendrimers are usually synthesized according to methods allowing for the control of the dendrimer structure at every stage. The dendrimeric structures are primarily synthesized by one of two different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building the dendrimer inwardly, and eventually attaching the structure to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20;20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents are linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO 2009/046446, WO 2015168347, WO 2016025745, WO 2016025741, WO 2019094952, and U.S. Pat. No. 8,889,101.

### B. Dendrimers Modified With Galactose (Gal)

It has been established that hydroxyl-terminated dendrimers conjugated with galactose (“Gal”) molecules selectively accumulate within hepatocyte cells and can be used to selectively deliver therapeutic, prophylactic or diagnostic agents to hepatocytes in the liver. Compositions of dendrimers modified by addition of one or more galactose moieties to the dendrimer (“D-Gal”) are described. In some embodiments, galactosylated hydroxyl-terminated dendrimers selectively target and internalize in hepatocytes in vitro and in vivo; and/or selectively accumulate within hepatocyte cells through multivalent binding with asialoglycoprotein receptors (ASGPR).

Significant breakthroughs in the field of nanomedicine have led to development of nanocarriers for targeted drug delivery to the liver, but a majority of them does not achieve the desired hepatocyte-specific localization in effective concentrations. In many reports, researchers even fail to distinguish between the non-selective accumulation of nanotherapeutics in the whole liver tissue versus specifically to the liver hepatocytes. The issue is that only a few nanoparticles are actually accumulating within the liver in a meaningful way, as they are cleared from circulation by both Kupffer cells (liver macrophages), through the mononuclear phagocyte system, and also from hepatocytes through hepatobiliary clearance as part of the reticuloendothelial system. The function of the reticuloendothelial system is critical to the removal of toxins and foreign bodies from the bloodstream, but proves a major hindrance to the delivery of drugs to hepatocytes specifically. It has been established that targeted nanomedicine using D-Gal nanoparticles overcomes hepatobiliary clearance by incorporating ligands to the nanoparticles’ surfaces that actively bind with asialoglycoprotein receptors (ASGPR) expressed on hepatocytes.

### I. ASGP-R Binding

Compositions of dendrimers conjugated with galactose target and localize within hepatocytes via interaction with asialoglycoprotein receptors (ASGPR) on the surface of hepatocyte cells. Therefore, compositions of D-Gal facilitate uptake via ASGPR binding in vivo.

Active targeting via ASGP-R, which is exclusively expressed on mammalian hepatocytes (~500,000 copies/cell), enables hepatocyte specific nanocarriers to attain enhanced drug distribution to these cells.

ASGP-R is a C type lectin that can specifically recognize ligands with a terminal galactose, glucose, or N-acetylgalactosamine (GalNAc). There have been reports where therapeutic molecules including small drug molecules, proteins scaffolds or antibodies are conjugated to galactose, GalNAc, lactose, or glucose monomers, or a small cluster of carbohydrates to target liver hepatocytes. Early on it became evident that monovalent binding of carbohydrates with proteins is often weak, in the millimolar range, and is not able to retain when facing rapid systemic blood flow. The multivalent binding affinity of (e.g., trivalent and tetravalent) carbohydrate constructs to ASGP-R is 100-1000 fold stronger compared to monovalent ligands, due to the glyco-cluster effect. There are several examples in the literature where commercially available nanoparticles have been modified partially or fully on the surface with carbohydrate units to increase their liver uptake. To date, no dendrimer-based nanoparticles for hepatocytes targeting through ASGPR have been reported showing liver uptake of ~20% injected dose within one hour of systemic administration with 85% of the hepatocytes positive for dendrimer, and with the dendrimer clearing intact from the rest of the body.

There are four categories of hepatocytes targeting systems:

1) Hepatocytes targeting systems through ASGPR are the cluster molecules, where triantennary N-acetyl galactosamine molecules are directly attached to one molecule of Si-RNA. These molecules lack the multivalency dendrimers offer for the covalent conjugation of the drugs for targeted delivery and release in the hepatocytes. (Prakash TP et al., Nucleic Acids Res, 42(13):8796-807 (2014); Prakash TP et al., J Med Chem, 59(6):2718-2733 (2016); Wang Y et al., Expert Opin Drug Metab Toxicol, 15(6):475-485 (2019)).

2) Polymers conjugated to galactosamine for hepatocytes targeting. The size of these polymeric nanoparticles are >50 nm and zeta potential is either highly positive or negative, not neutral such as Gal24 and D4-Gal dendrimer constructs (~5 nm) shown in the Examples. (Wang H et al., J Control Release, 166(2):106-14 (2013); Paolini M et al., Int J Nanomedicine, 12:5537-5556 (2017)). These are not ideal for hepatocyte internalization due to their size (~50-100 nm, compared to dendrimers ~4 nm).

3) Generation 5 (G5) PAMAM-NH₂ conjugated to galactose for the delivery of plasmid. No quantitative uptake numbers were reported for hepatocytes. These amine dendrimers are also cytotoxic due to their cationic nature and are not suitable for systemic drug delivery.

4) Nanoparticles, other than dendrimers, taking advantage of hepatocytes targeting through mechanisms different to targeting ASGPR.

A galactose-based generation 2 (G2) glycodendrimer construct (“D2-Galactose-24”, or “D2-GAL-24”) has been designed and developed having strong recognition to target hepatocytes following systemic administration, for the specific delivery of therapeutics to hepatocytes to diagnose, treat, and/or prevent one or more liver diseases or disorders while displaying minimum systemic uptake and side effects/toxicities.

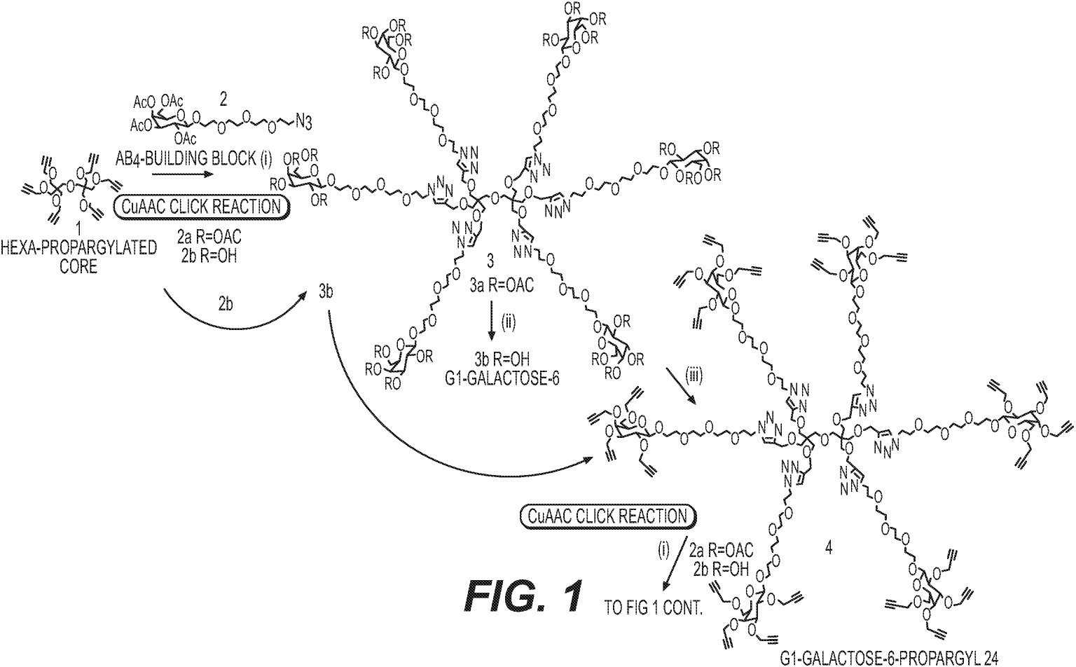

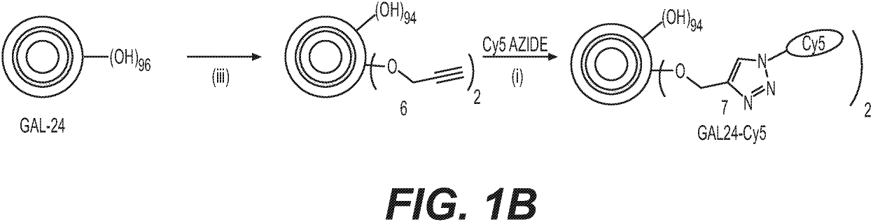

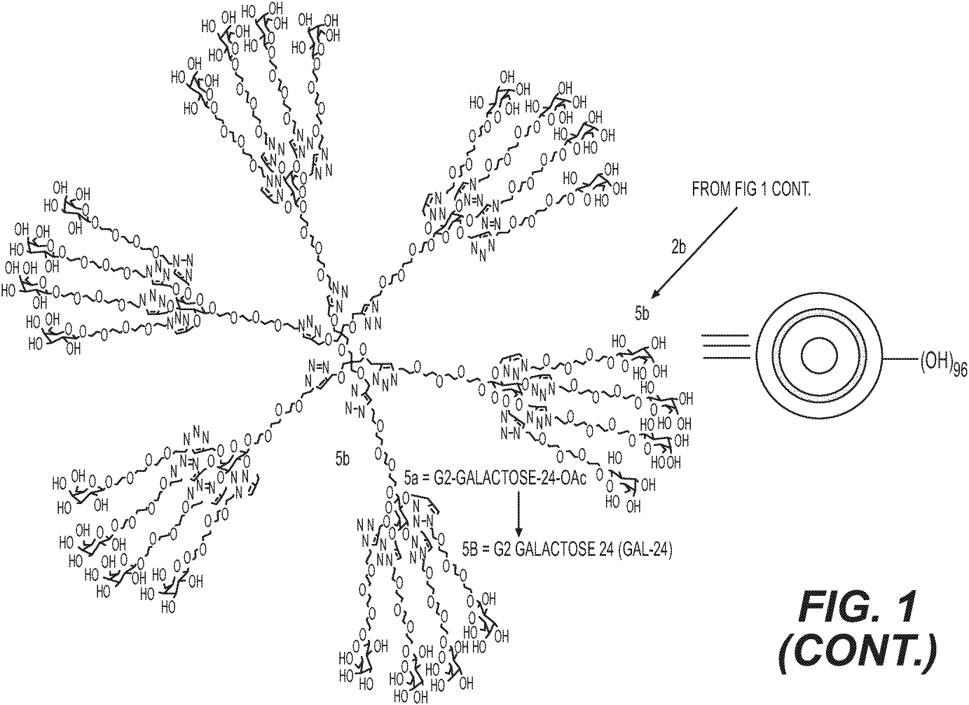

In some embodiments, the galactosylated dendrimer is a dendrimer made of galactose and oligoethylene glycol building blocks, of generation 1 (24 OH terminal groups), generation 2 (96 OH terminal groups), generation 3 (384 OH terminal groups), or generation 4 (1536 OH terminal groups). In one embodiment, the galactosylated dendrimer is a dendrimer composed of galactose and oligoethylene glycol building blocks (GAL-24) as shown in FIG. 1A. The selective functionalization at the anomeric position of galactose leads to the generation of orthogonal hypermonomers and building blocks. Synthesis of GAL-24 with the construction of hexapropargylated core and AB4 β-Gal-PEG4-azide building blocks by performing glycosidic linkages at C1 with orthogonal PEG4 linkers (FIG. 1A). The final dendrimer (D2-GAL-24) with 24 galactose and 96 OH groups at generation 2 is depicted in FIG. 1A. This galactosylated dendrimer is a generation 2 dendrimer (D2), where 24 galactose units include the outer layer and six galactose units are embedded in the dendrimer backbone, connected through tetraethylene glycol (PEG4) units.

The multivalent nature of this galactose-based dendrimer allowed for enhanced binding to ASGP-R with an affinity ~100-fold lower than free β-d-galactose. Binding to ASGP-R resulted in high levels of D2-GAL-24 internalization in HEPG2 cells as well as in hepatocytes in vivo. The mechanism of cellular internalization was determined to be majorly receptor-mediated endocytosis, as evidenced by competitive uptake with free galactose as well as blocking the assembly of clathrin. The internalized D2-GAL-24 was found to be non-toxic to a number of common human cell lines in vitro and showed no obvious signs of toxicity to clearance organs in vivo, clearing intact from the body. Upon systemic administration in healthy mice, D2-GAL-24 localized mostly in the liver having ~20% injected dose (ID) at one hour after administration, and ~2% ID 48 hours later. All off-target organs expressed less than 0.2% ID of D2-GAL-24 signal 48 hours after administration, indicating rapid clearance from the rest of the body. The majority of D2-GAL-24 in the liver was within hepatocytes, as determined by both confocal imaging and flow cytometry. Over 85% of primary mouse hepatocytes were observed containing fluorescently-labeled D2-GAL-24 as opposed to less than 10% of non-parenchymal cells observed as positive for D2-GAL-24. This hepatocyte-specific in vivo delivery was maintained in both a mouse model of severe acetaminophen poisoning-induced hepatic necrosis, and a rat model of non-alcoholic steatohepatitis, where confocal imaging showed that D2-GAL-24 strongly co-localized with hepatocyte signal.

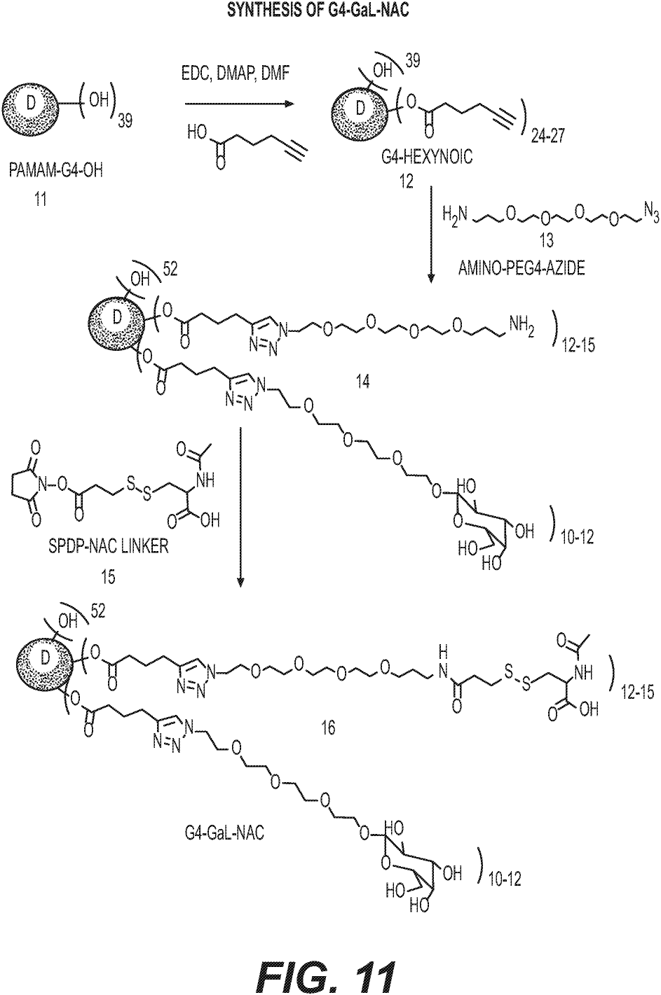

In one embodiment, the galactose-modified dendrimer is a generation 4 hydroxyl-terminated PAMAM dendrimer, modified with 10-12 galactose molecules (D4-Gal-10/12) on the surface, as shown in FIG. 11. It has been shown in the Examples that the surface galactose sugars create a multivalent binding effect to ASGPR, allowing the galactosylated dendrimer to selectively target and internalize in hepatocytes in vitro and in vivo. D4-Gal has shown to be a highly specific delivery vehicle in selectively targeting hepatocytes. There are nanosystems with stronger binding affinities to ASGPR, higher accumulation in the liver, and a longer demonstrated residence time in the liver; however, no compound has yet been developed that does all three along with specific targeting for hepatocytes, not just liver tissue. Such specificity was demonstrated in the Examples through flow cytometry of primary liver cells.

The D4-Gal conjugate has a high affinity for the asialoglycoprotein receptor expressed on liver macrophages, resulting in both increased uptake in HEPG2 cells in vitro and liver tissue in vivo. Localization in the liver is highly specific to hepatocytes, with D4-Gal present in hepatocytes at a ratio of 25:1 compared to non-parenchymal cells of the liver. Furthermore, there is rapid off-target clearance of D4-Gal with no organ except the kidneys containing more than 0.1% of the original injected dose after just 48 hours whereas D4-Gal was still visible in hepatocytes a week after injection. This preferential liver uptake is maintained in both a mouse model of acetaminophen induced liver failure and a rat model of non-alcoholic steatohepatitis. Gal-D4-NAC was synthesized with an additional 15 molecules of N-acetyl cysteine attached to the dendrimer surface via glutathione sensitive linkers for application in a mouse model of severe acetaminophen poisoning (FIG. 11). A single intravenous dose of Gal-D4-NAC at 100 mg/kg on a NAC basis provided dramatic improvement to liver function and structure by reducing serum aminotransferase levels and restoring hepatocellular organization as seen through histology. Free NAC at an equivalent dose was unable to have any effect on liver health compared to untreated animals, indicating that Gal-D4-NAC is able to increase the therapeutic window for the thousands of patients that die or require liver transplants due to extreme acetaminophen overdose or delayed treatment. In some embodiments, conjugation of galactose molecules through one or more surface groups occurs via about 1%, 2%, 3%, 4%, 5%,, 6%, 7%, 8%, 9%, or 10% of the total available surface functional groups, preferably hydroxyl groups, of the dendrimers prior to the conjugation. In other embodiments, the conjugation of galactose molecules occurs on less than 5%, less than 10%, less than 15%, less than 20%, less than 25%, less than 30%, less than 35%, less than 40%, less than 45%, less than 50% of total available surface functional groups of the dendrimers prior to the conjugation. In preferred embodiments, dendrimers are conjugated to an effective amount of galactose molecules for binding to ASGPR and/or targeting to hepatocytes, whilst conjugated to an effective amount of active agents to treat, prevent, and/or image the liver disease or disorder.

While galactose has a strong affinity for ASGPR, a common ligand with improved binding affinity is N-acetylgalactosamine (GalNAc), which has a micromolar scale monovalent binding affinity. GalNAc conjugates are widely used, but some have reported hepatotoxicities as well as off-target binding. Accordingly, in further embodiments, the dendrimers are not conjugated to N-Acetylgalactosamine (GalNAc).

### C. Dendrimer Complexes

Galactosylated dendrimers have tremendous translational potential, as the versatility of conjugation chemistries to the hydroxyl surface groups allows for the attachment of small molecules, imaging agents, and potentially small biologics such as siRNA regardless of the payload’s charge or aqueous solubility. Dendrimers modified with galactose (dendrimer-galactose, or D-Gal) can include one or more therapeutic or prophylactic agents complexed, covalently conjugated, or intra-molecularly dispersed or encapsulated with the dendrimer. Conjugation of one or more agents to the dendrimer component of a dendrimer-Gal complex can occur prior to, at the same time as, or subsequent to conjugation of the dendrimer with the galactose. Compositions and methods for conjugating agents with dendrimers are known in the art, and are described in detail in U.S. Published Application Nos. US 2011/0034422, US 2012/0003155, and US 2013/0136697.

In some embodiments, one or more agents are covalently attached to the dendrimer component of the dendrimer-galactose (D-Gal). In some embodiments, D-Gal complexes include one or more active agents conjugated or complexed with the D-Gal via one or more linking moieties. In further embodiments, the linking moieties incorporate or are conjugated with one or more spacer moieties. The linking and/or spacer moieties can be cleavable, for example, by exposure to the intracellular compartments of hepatocyte cells in vivo. The active agent and/or targeting moiety can be either covalently attached or intra-molecularly dispersed or encapsulated. The galactosylated dendrimer is preferably a PAMAM dendrimer from generation 0, up to generation 10 (D0-D10-Gal), having hydroxyl terminations. In preferred embodiments, the D-Gal is linked to agents via a spacer ending in disulfide, ester or amide bonds.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more active agents are encapsulated, associated, and/or conjugated to the dendrimer component of the dendrimer-galactose complex at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of a dendrimer to an active agent occurs prior to conjugation of the dendrimer with galactose. In some embodiments, conjugation of active agents and/or linkers to dendrimer-galactose occurs through one or more surface and/or interior groups. Thus, in some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation and/or the modification with galactose and/or active agents. In preferred embodiments, dendrimer complexes retain an effective amount of surface functional groups for modification with galactose for targeting to hepatocytes, whilst conjugated to an effective amount of agents for treat, prevent, and/or image the disease or disorder.

### D. Coupling Agents and Spacers

Dendrimer complexes can be formed of therapeutically agents or compounds conjugated or attached to a dendrimer, a dendritic polymer or a hyperbranched polymer. Optionally, the active agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, one or more spacers/linkers between a dendrimer and an agent are added to achieve desired and effective release kinetics in vivo.

### I. Coupling Agents

In some embodiments, the active agents are attached to the dendrimer via a linking moiety that is designed to be cleaved in vivo. The linking moiety can be designed to be cleaved hydrolytically, enzymatically, or combinations thereof, so as to provide for the sustained release of the agents in vivo. Both the composition of the linking moiety and its point of attachment to the agent, are selected so that cleavage of the linking moiety releases either an active agent, or a suitable prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, —CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers.

### Ii. Spacers

In certain embodiments, the linking moiety includes one or more of the organic functional groups described above in combination with a spacer group. The term “spacers” includes compositions used for linking a therapeutically agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

In preferred embodiments, the attachment of the active to the dendrimer occurs via an appropriate spacer that provides a disulfide bridge between the active agent and the dendrimer. In one embodiment, the dendrimer-galactose complexes rapidly release the agent by thiol exchange reactions, under the reduced conditions found in vivo.

The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the anti-inflammatory agent and the dendrimers. In some embodiments, the spacer is chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, bis-maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl and thiol terminations.

### E. Therapeutic, Prophylactic and Diagnostic, Agents

D-Gal complexes deliver active agents selectively to hepatocyte cells in vivo. Agents to be included in the D-Gal complexes to be delivered to hepatocytes can be proteins or peptides, sugars or carbohydrate, nucleic acids or oligonucleotides, lipids, small molecules (e.g., molecular weight less than 2,000 Dalton, preferably less than 1,500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the agent is a therapeutic antibody.

Dendrimers have the advantage that multiple therapeutic, prophylactic, and/or diagnostic agents can be delivered with the same dendrimers. One or more types of agents can be encapsulated, complexed or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment.

Active agents can include those that alleviate or treat one or more symptoms of one or more liver disease/disorders. Exemplary active agents are anti-inflammatory agents and anti-oxidant agents.

### I. Therapeutic and Prophylactic Agents

The D-Gal complexes include one or more therapeutic, prophylactic, or prognostic agents that are complexed or conjugated to the dendrimers. Representative therapeutic agents include, but are not limited to, anti-inflammatory agents, antioxidants, anti-infectious agents, and combinations thereof.

In some embodiments, the compositions include one or more anti-inflammatory agents. Anti-inflammatory agents reduce inflammation and include steroidal and non-steroidal drugs.

A preferred anti-inflammatory is an antioxidant drug including N-acetylcysteine. Preferred NSAIDS include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

Representative small molecules include steroids such as methyl prednisone, dexamethasone, non-steroidal anti-inflammatory agents including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, such as baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone.

Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukins 12 and 23.

In some embodiments, the anti-inflammatory drug is a synthetic or natural anti-inflammatory protein. Antibodies specific to select immune components can be added to immunosuppressive therapy. In some embodiments, the anti-inflammatory drug is an anti-T cell antibody (e.g., anti-thymocyte globulin or Anti-lymphocyte globulin), anti-IL-2Rα receptor antibody (e.g., basiliximab or daclizumab), or anti-CD20 antibody (e.g.., rituximab).

Many inflammatory diseases may be linked to pathologically elevated signaling via the receptor for lipopolysaccharide (LPS), toll-like receptor 4 (TLR4). There has thus been great interest in the discovery of TLR4 inhibitors as potential anti-inflammatory agents. Recently, the structure of TLR4 bound to the inhibitor E5564 was solved, enabling design and synthesis of new TLR4 inhibitors that target the E5564-binding domain. These are described in U.S. Pat. No. 8,889,101, the contents of which are incorporated by reference. As reported by Neal, et al., PLoS One. 2013; 8(6): e65779e, a similarity search algorithm used in conjunction with a limited screening approach of small molecule libraries identified compounds that bind to the E5564 site and inhibit TLR4. The lead compound, C34, is a 2-acetamidopyranoside (MW 389) with the formula C₁₇H₂₇NO₉, which inhibits TLR4 in enterocytes and macrophages in vitro, and reduces systemic inflammation in mouse models of endotoxemia and necrotizing enterocolitis. Thus, in some embodiments, the active agents are one or more TLR4 inhibitors. In preferred embodiments, the active agents are C34, and derivatives, analogues thereof.

In one embodiment, the therapeutic or prophylactic agent is N-acetyl cysteine.

### Ii. Diagnostic Agents

In some cases, the agents delivered to hepatocytes via D-Gal are diagnostic agents. Examples of diagnostic agents that can be delivered to hepatocytes by D-Gal complexes include paramagnetic molecules, fluorescent compounds, magnetic molecules, and radionuclides, x-ray imaging agents, and contrast media. Examples of other suitable contrast agents include gases or gas emitting compounds, which are radioopaque. D-Gal complexes can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thüicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer complex include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F), e.g., 2-deoxy-2-¹⁸F-fluoro-β-D-glucose (¹⁸F-FDG).

In further embodiments, a singular D-Gal complex composition can simultaneously treat and/or diagnose a disease or a condition at one or more locations in the body.

### III. Pharmaceutical Formulations

Pharmaceutical compositions including galactosylated dendrimers and one or more active agents e.g., N-acetyl cysteine, may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain one or more galactosylated dendrimer complexes in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington’s Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions of D-Gal are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions of D-Gal are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions of D-Gal formulated for administration by parenteral (intramuscular, intraperitoneal, intravenous (IV) or subcutaneous injection) and enteral routes of administration are described.

### A. Parenteral Administration

The compositions of D-Gal can be administered parenterally. The phrases “parenteral administration” and “administered parenterally” are art-recognized terms, and include modes of administration other than enteral and topical administration. The dendrimers can be administered parenterally, for example, by subdural, intravenous, intrathecal, intraventricular, intraarterial, intra-amniotic, intraperitoneal, or subcutaneous routes.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer’s dextrose, dextrose and sodium chloride, lactated Ringer’s and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer’s dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of D-Gal can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer’s dextrose, dextrose and sodium chloride, lactated Ringer’s and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer’s dextrose. In general, water, saline, aqueous dextrose and related sugar solutions are preferred liquid carriers. These can also be formulated with proteins, fats, saccharides and other components of infant formulas.

In preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Use

It has been established that galactose-modified dendrimer compositions selectively bind to asialoglycoprotein receptors (ASGPR) on hepatocyte cells. The efficient binding to the ASGPR receptors directs selective internalization of the dendrimer-galactose within the hepatocyte. Methods of using galactosylated dendrimer compositions are described. Methods of selective delivery of active agents to hepatocytes are also provided. In some embodiments, the methods of using the galactosylated dendrimer compositions selectively deliver one or more active agents to hepatocytes in vivo. Methods for selective delivery, accumulation, and intracellular release of one or more active agents to hepatocytes for the treatment, prevention and diagnosis of liver diseases or disorders are described.

### A. Methods of Treating Liver Disorders and Diseases

Methods of using dendrimer-galactose compositions for treating or preventing one or more liver diseases or disorders in a subject are described.

Galactosylated dendrimer compositions, including one or more active agents to treat or prevent a liver disease or disorder can be administered to a subject to treat, prevent, and/or diagnose one or more symptoms of one or more liver disorders and/or diseases in the subject. The methods can include the step of identifying and/or selecting a subject in need thereof. The compositions and methods are also suitable for prophylactic use.

Methods for treating or preventing one or more symptoms of one or more liver disorders and/or diseases include administering to the subject galactosylated dendrimers complexed, covalently conjugated, or intra-molecularly dispersed or encapsulated with one or more therapeutic or prophylactic agents, in an amount effective to treat, alleviate or prevent one or more symptoms of one or more liver disorders and/or diseases. In preferred embodiments, the D-Gal compositions including one or more antioxidant agents and/or one or more anti-inflammatory, or formulations thereof are administered in an amount effective to treat or prevent one or more symptoms of one or more liver disorders and/or diseases, for example, reducing lobular inflammation in the liver. D-Gal complexes may also include a targeting agent, but as demonstrated by the examples, these are not required for delivery to healthy and/or injured hepatocytes in the liver.

In some embodiments, the D-Gal complexes include an agent that is attached or conjugated to dendrimers, which are capable of preferentially releasing the drug intracellularly under the reduced conditions found in vivo. The agent can be either covalently attached or intra-molecularly dispersed or encapsulated. The amount of D-Gal complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer. In preferred embodiments, methods for treating or preventing one or more symptoms of a liver disease and/or disorder in a subject in need thereof include administering to the subject a formulation including galactosylated dendrimers complexed to, covalently conjugated to, or having intra-molecularly dispersed or encapsulated therein one or more therapeutic or prophylactic agents. In one embodiment, methods for treating or preventing one or more liver disorders and/or diseases include administering to the subject compositions including galactose-modified hydroxyl terminated dendrimers of generation 4, generation 5, generation 6, generation 7, or generation 8 covalently conjugated to one or more anti-inflammatory agents (e.g., N-acetyl cysteine), in an amount effective to treat or prevent one or more symptoms of one or more liver disorders and/or diseases. In preferred embodiments, the formulation is administered in an amount effective to treat, alleviate or prevent one or more symptoms of a liver disease and/or disorder selected from inflammatory liver diseases, non-alcoholic steatohepatitis, drug-induced liver failure, hepatitis, liver fibrosis, liver cirrhosis, or combinations thereof.

### B. Liver Disorders and Diseases to Be Treated

Dendrimer-galactose (D-Gal) compositions are effective for treating or ameliorating one or more symptoms of a liver disease, or disorder, such as acute or chronic liver diseases. Exemplary indications that can be treated include, but are not limited to, acute liver failure (acute hepatitis, fulminant hepatitis), e.g., resulting from neoplastic infiltration, acute Budd-Chiari syndrome, heatstroke, mushroom ingestion, metabolic diseases such as Wilson’s disease, or associated with viral liver disease such as caused by herpes simplex viruses, cytomegalovirus, Epstein-Barr virus, parvoviruses, hepatitis viruses (e.g., hepatitis A, hepatitis E, hepatitis D+B infections), or drug-induced liver injury, including rifampicin-induced hepatotoxicity, acetaminophen-induced hepatotoxicity, recreational-drug induced toxicity such as by 3,4-methylenedioxy-N-methylamphetamine (MDMA, also known as ecstasy), or cocaine-induced toxicity, acute ischemic hepatocellular injury, or hypoxic hepatitis, or resulting from traumatic liver injury. The methods can treat and prevent any hyperacute, acute and subacute liver disease defined by the occurrence of encephalopathy, coagulopathy and jaundice in an individual with a previously normal liver.

Symptoms and clinical manifestations of acute liver disease include jaundice and encephalopathy, and impaired liver function (e.g., loss of metabolic function, decreased gluconeogenesis leading to hypoglycemia, decreased lactate clearance leading to lactic acidosis, decreased ammonia clearance leading to hyperammonemia, and reduced synthetic capacity leading to coagulopathy). Acute liver diseases and disorders are often associated with multiple systemic manifestations, including immunoparesis contributing to high risk of sepsis; systemic inflammatory responses, with high energy expenditure or rate of catabolism; portal hypertension; kidney dysfunction; myocardial injury; pancreatitis (particularly in acetaminophen-related disease); inadequate glucocorticoid production in the adrenal gland contributing to hypotension; and acute lung injury, leading to acute respiratory distress syndrome.

The methods of treatment can also include the step of identifying and selecting a subject in need of treatment, or a subject who would benefit from administration with the D-Gal compositions. In some embodiments, the subject has been medically diagnosed as having an acute liver disease or disorder by exhibiting clinical (e.g., physical) symptoms of the disease. In other embodiments, the subject has been medically diagnosed as having a sub-acute or chronic liver disease or disorder by exhibiting clinical (e.g., physical) symptoms, which are indicative of an increased risk or likelihood of developing acute liver disease. Therefore, in some embodiments, formulations of the disclosed D-Gal compositions are administered to a subject prior to a clinical diagnosis of acute liver disease.

In some embodiments, the D-Gal compositions are administered in an amount effective to inhibit or reduce serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality.

In some embodiments, the methods treat or prevent hepatocellular carcinoma (HCC). HCC is the most common primary liver malignancy and is a leading cause of cancer-related death worldwide.

In preferred embodiments, the methods treat or prevent non-alcoholic steatohepatitis, liver fibrosis associated with non-alcoholic steatohepatitis (NASH). In further preferred embodiments, the methods treat or prevent severe acetaminophen (APAP) poisoning.

Methods to treat and/or prevent one or more symptoms of APAP poisoning or NASH typically include administering to a subject in a need thereof an effective amount of a composition including galactose-modified hydroxyl terminated dendrimers and one or more agents to treat and/or alleviate one or more symptoms associated with APAP poisoning or NASH. In one embodiment, the dendrimer compositions include galactose-modified hydroxyl terminated PAMAM dendrimers of generation 4, generation 5, or generation 6 covalently conjugated to one or more anti-inflammatory agents.

### C. Dosage and Effective Amounts

Dosage and dosing regimens are dependent on the severity and location of the disorder or injury and/or methods of administration, and can be determined by those skilled in the art. A therapeutically effective amount of the dendrimer composition used in the treatment of liver disorders and/or diseases is typically sufficient to reduce or alleviate one or more symptoms of liver disorders and/or diseases.

Preferably, the active agents do not target or otherwise modulate the activity or quantity of healthy cells not within or associated with the diseased/damaged tissue, or do so at a reduced level compared to cells associated with the diseased/damaged liver. In this way, by-products and other side effects associated with the compositions are reduced.

Administration of the D-Gal compositions leads to an improvement, or enhancement, function in an individual with a liver disease, injury, or disorder.

The actual effective amounts of D-Gal complex can vary according to factors including the specific agent administered, the particular composition formulated, the mode of administration, and the age, weight, condition of the subject being treated, as well as the route of administration and the disease or disorder. In some embodiments, dosage ranges suitable for use are between about 0.01 and about 100 mg/kg body weight, inclusive; between about 0.1 mg/kg and about 10 mg/kg, inclusive; between from about 0.5 mg and about 5 mg/kg body weight, inclusive. Generally, for intravenous injection or infusion, the dosage may be lower than for oral administration.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill in the art can easily determine optimum dosages, dosing methodologies and repetition rates. Optimum dosages can vary depending on the relative potency of individual pharmaceutical compositions, and can generally be estimated based on EC_{50S} found to be effective in in vitro and in vivo animal models.

Dosage forms of the pharmaceutical composition including the dendrimer compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose. In some embodiments, the dosage unit suitable for use are (assuming the weight of an average adult patient is 70 kg) between 5 mg/dosage unit and about 7000 mg/ dosage unit, inclusive; between about 35 mg/ dosage unit and about 2800 mg/ dosage unit, inclusive; and between about 70 mg/ dosage unit and about 1400 mg/ dosage unit, inclusive; and between about 140 mg/ dosage unit and about 700 mg/ dosage unit, inclusive.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly or yearly dosing.

In some embodiments, dosages are administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### D. Combination Therapies and Procedures

The D-Gal compositions can be administered alone or in combination with one or more conventional therapies. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition more than one active agent. Such formulations typically include an effective amount of an agent targeting the site of treatment. The additional active agent(s) can have the same or different mechanisms of action. In some embodiments, the combination results in an additive effect on the treatment of the liver condition. In some embodiments, the combinations result in a more than additive effect on the treatment of the disease or disorder.

The additional therapy or procedure can be simultaneous or sequential with the administration of the dendrimer composition. In some embodiments, the additional therapy is performed between drug cycles or during a drug holiday that is part of the compositions dosage regime.

In some embodiments, the compositions and methods are used prior to or in conjunction, subsequent to, or in alternation with treatment with one or more additional therapies or procedures.

### E. Controls

The therapeutic result of the dendrimer complex compositions including one or more active agents can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art. In some embodiments, an untreated control subject suffers from the same acute liver disease or condition as the treated subject.

### V. Kits

The compositions of D-Gal can be packaged in kit. The kit can include a single dose or a plurality of doses of a composition including one or more active agents such as anti-inflammatory agents, encapsulated in, associated with, or conjugated to a galactosylated dendrimer, and instructions for administering the compositions. Specifically, the instructions direct that an effective amount of the D-Gal composition be administered to an individual with a particular liver disease/disorder as indicated. The composition can be formulated as described above with reference to a particular treatment method and can be packaged in any convenient manner.

The present invention will be further understood by reference to the following non-limiting examples.

### EXAMPLES

### Example 1: Synthesis and Characterization of GAL-24

### Materials and Methods

Carbohydrates are biologically significant scaffolds which have gamered a lot of interest from the researchers working in the development of nanoparticle based drug delivery systems due to their outstanding advantages such as inherent biocompatibility, structural rigidity, high water solubility, easy availability, and low cost. Above all, the selective functionalization at the anomeric position leads to the generation of orthogonal hypermonomers and building blocks with ease which leads to the rapid synthesis of diverse glycol-constructs. Galactose interacts with asialoglycoprotein receptors (ASGP-R) through OH groups at C3 and C4 positions. Modifications at C1 through glycosylation of galactose maintains high binding affinity to ASGP-R.

The synthesis of GAL-24 began with the construction of hexapropargylated core (1) and AB4 β-Gal-PEG4-azide building blocks (peracetylated 2a and deprotected 2b) by performing glycosidic linkages at C1 with orthogonal PEG4 linkers using published literature procedures in multiple gram scale quantities with ease and characterized them using NMR spectroscopy. β-Gal-PEG4-azide building blocks are also commercially available and can be directly used as received. GAL-24 dendrimer was synthesized via two synthetic routes, using protected and unprotected AB4 building blocks as represented in FIG. 1A (red and black arrows). Although both synthetic strategies yielded the same dendrimer product in good yields, high purity and reproducibility, the use of unprotected orthogonal approach is an accelerated route which yielded the final dendrimer with 24 galactose and 96 OH groups at generation 2 very rapidly in just 3 steps (FIG. 1A, red arrows). The reason for constructing the dendrimer using two strategies is to overcome the complexity associated with the characterization of the macromolecules. Although ¹H NMR is the most convincing and widely used analytical tool to characterize the organic constructs, the characterization of dendrimers using NMR is difficult and complex due to the presence of huge number of interfering proton peaks in narrow region and catching a defect in the structure is like finding a needle in the haystack. This further requires the need for addition characterization tools to confirm the structure. Failure to assess the absolute structure and composition of nanoparticles is a major pitfall in their clinical translation. With the thoughtful use of protecting building blocks, it has been clearly demonstrated the success of chemical reactions at each step of dendrimer synthesis by the appearance and disappearance of acetate protons which did not interfere with the peaks of dendrimer protons.

To construct the dendrimer via protected route, the CuAAC click reaction was performed between the hexa-propargylated core (1) and the peracetylated β-Gal-PEG4-azide (2a) using classical click reagents, a catalytic amount of copper sulfate pentahydrate and sodium ascorbate to achieve G1-Galactose-6-OAc (3a, FIG. 1A). The disappearance of propargyl protons at δ2.42 ppm and the appearance of triazole protons at δ7.7 ppm confirmed the successful completion of click reaction. In addition, the acetate protons appear in between δ1.95-2.20 ppm in the proton NMR. The treatment of peracetylated dendrimer G1-Galactose6-OAc (3a) under typical Zemplén conditions provided G1-Galactose-6 (3b). Complete acetate deprotection was confirmed by the absence of acetate protons in ¹H NMR. The click reaction of the core (1) with unprotected β-Gal-PEG4-azide (2b) yielded the same product (3b) with ease. The terminal OH groups in G1 dendrimer 3b were further conveniently propargylated using NaH and propargyl bromide to obtain G1-Galctose-6-propargyl24 (4). The successful propargylation was confirmed by the appearance of peak corresponding to acetylene protons at δ2.4 ppm in NMR. The active terminal alkyne groups in dendrimer 4 were further employed in CuAAC click reaction with peracetylated β-Gal-PEG4-azide (2a) to produce G2-Galactose-24-OAc (5a). Once again, ¹H NMR clearly confirmed the formation of the product where peaks corresponding to 24 propargyl protons completely vanished with the appearance of triazole protons in between δ8.0-7.5 ppm and acetate protons in between δ2.1-1.8 ppm. The deprotection of acetate groups was achieved via Zemplén transesterification reaction to yield the final dendrimer GAL-24 (5b). Same product (5b) was synthesized by the reaction of compound 4 with unprotected β-Gal-PEG4-azide (2b). The final dendrimer (5b) is highly water soluble and was purified using tangential flow filtration through 1 kDa cassette.

### Results

The disappearance of acetate peaks in the proton NMR convinced the formation of product. All the intermediates and final dendrimer were characterized via NMR, Mass and HPLC. HPLC chromatogram of GAL-24 showed a retention time of 8.3 minutes while G1-Galactose-6 had a retention time at 8.04 minutes. The HPLC purity of GAL-24 is >99%. Additional confirmation was obtained by MALDI-ToF which showed a peak at 12,870 Da. The size and zeta potential of GAL-24 was measured using dynamic light scattering. GAL-24 demonstrated a size of approximately 5.3 nm with nearly neutral zeta potential of approximately 7.9 mV. In comparison to conventional dendrimer synthesis, this strategy employed here is straightforward and highly efficient which quickly leads to the formation of perfectly defined dendrimer in a strikingly easy way.

To study the in vitro and in vivo hepatocyte uptake of GAL-24 via confocal microscopy and fluorescence spectroscopy, a near infra-red dye cyanine 5 (Cy5) was attached on the surface of GAL-24 through a non-cleavable ether linkage. 2-3 OH groups were modified on the surface of GAL-24 (5b) by reacting with propargyl bromide in the presence of sodium hydride to afford compound 6 (FIG. 1B). The ¹H NMR showed the presence of propargyl H at δ2.7 ppm. The dendrimer 6 with 2-3 propargyl groups on the surface was reacted with azide terminating Cy5 using CuAAC click reaction to obtain fluorescently labelled GAL24-Cy5 7. The success of cy5 attachment was confirmed by the presence of Cy5 protons in proton NMR spectrum. The purity of GAL24-Cy5 was >97% by HPLC and HPLC clearly showed a shift in the retention time from 8.30 minutes to 9.95 minutes upon the conjugation of Cy5.

### Example 2: GAL-24 Binds to Asialoglycoprotein Receptor in Vitro Resulting in Hepatocellular Internalization Via Asialoglycoprotein Receptor-Mediated Endocytosis

### Materials and Methods

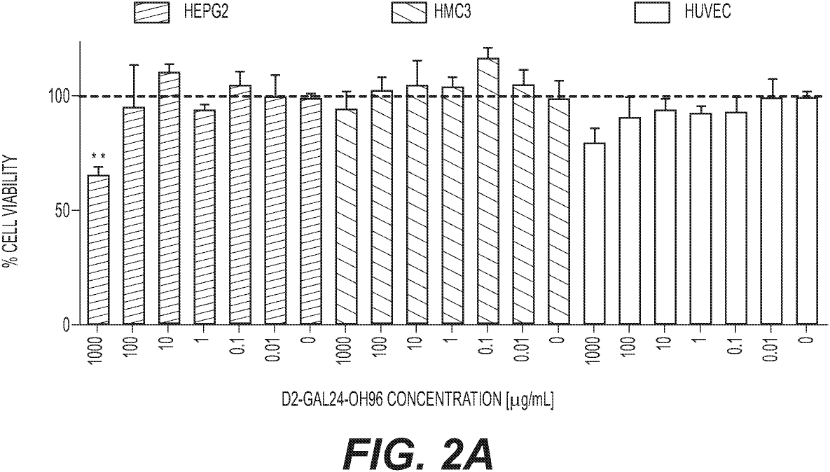

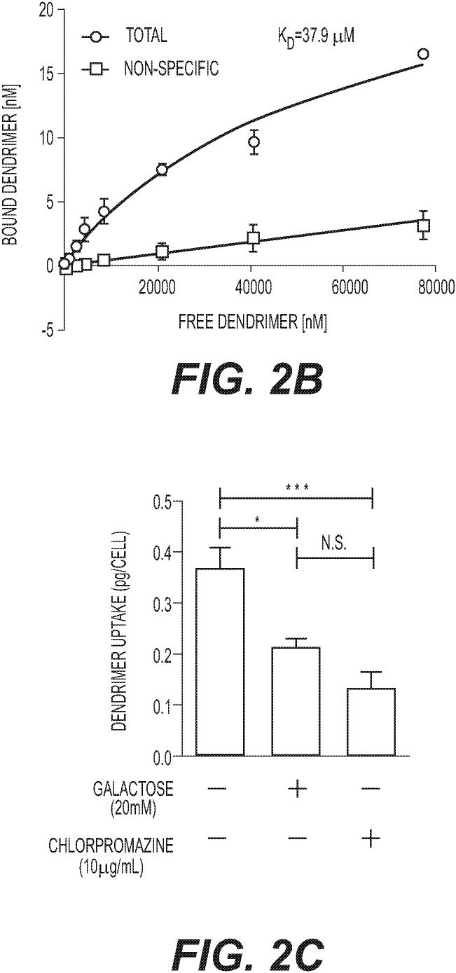

The baseline toxic effects were first assessed. Cell viability assays were performed in three cell lines: HEPG2 (human hepatocellular carcinoma), HMC3 (human microglia), and HUVEC (human umbilical vein endothelial cell). These three cell lines encompass the most likely candidates to take up the new particle in vivo, as other dendrimers have been shown to accumulate in microglia and macrophages as well as endothelial cells of blood vessels, and galactose targeting will bring GAL-24 additionally to hepatocytes. Each cell line was cultured with varying concentrations of GAL-24 up to 1000 µg/mL (FIG. 2A). HMC3 and HUVEC cells were viable through 1000 µg/mL as determined by one-way ANOVA with no significant effect of treatment (F(6,18)=0.8473, p>0.05, r²=0.2202, n=3 and F(6,19)=1.929, p>0.05, r²=0.3786, n=3 respectively). Viability of HEPG2 cells was reduced only at the highest treatment of 1000 µg/mL to 66% of untreated control cell viability (One-way ANOVA F(6,19)=3.910, p<0.05, r²=0.5525, n=3 followed by Student’s t-test with Dunnett’s correction p=0.0054 1000 µg/mL v. untreated). The decrease of viability only in the hepatocyte cell line could be due to increased accumulation in those cells from active transport into the cell via ASGP-R, and will not pose an issue in these studies as cells and animals will not be exposed to extremely high concentrations of GAL-24.

The multivalent effect of the GAL-24 on binding to ASGP-R was first investigated through a cell surface saturation experiment with HEPG2 cells. HEPG2 cells express ~76,000 ASGP-R per cell, providing sufficient receptors to bind the GAL-24 ligand. This method was chosen over surface plasmon resonance (SPR) studies due to the direct translatability of investigating cell-bound receptors in 3D as opposed to immobilized monolayers of receptor. Titrating GAL-24 concentration with a constant number of cells (and therefore receptors) allowed us to calculate K_{D} from the law of mass action using a modified GraphPad program for one site with non-specific binding.

### Results

Multiple replicates revealed the binding affinity of GAL-24 to ASGP-R to be 37.9 µM (FIG. 2B, 95% confidence interval 23.5-64.2 µM, r²=0.9554 for total binding, r²=0.8296 for non-specific binding, n=3). The K_{D} of GAL-24 is 100-fold lower than that of free β-d-galactose to ASGP-R (K_{D} ≈ 2-5 mM) potentially due to multivalent effects from the dendritic structure of GAL-24. Multivalency effects for surface receptor binding have been widely observed with dendrimer compounds previously as well as specifically to ASGP-R when nanocomplexes display multiple ligands for the receptor.

This strong binding of GAL-24 to ASGP-R could lead to enhanced internalization of GAL-24 into hepatocytes in culture. GAL-24 was found present at high levels intracellularly in HEPG2 human hepatocellular carcinoma cells after 24 hours of incubation (FIG. 2C). In healthy, confluent monolayers of HEPG2 cells incubated with 20 µg/mL (~2 µM) of GAL-24, 0.37 pg/cell of GAL-24 were extracted (n=8). This uptake could be significantly reduced by approximately 40% to 0.22 pg/cell (one-way ANOVA main effect of treatment F(2,18)=14.48, p<0.01, r²=0.6168, significantly decreased based on Student’s t-test with Tukey’s correction p=0.0106) upon pre- and co-treatment with free β-d-galactose at 10,000-fold higher concentration than GAL-24 (n=6). This indicates that the GAL-24 is most likely making use of the same entry pathway into the cell as free galactose, namely ASGP-R-mediated uptake.

The question remained if the uptake decrease observed with competitive β-d-galactose treatment was due to ASGP-R-mediated endocytosis or another cellular internalization mechanism. To this end, chlorpromazine, a small molecule endocytosis inhibitor known to interfere with the assembly of clathrin, was used, thus preventing receptor-mediated endocytosis in pathways where clathrin plays a role, like with ASGP-R-mediated endocytosis. When the HEPG2 cells were pre- and co- treated with 10 µg/mL chlorpromazine, uptake of GAL-24 was reduced to 0.13 pg/cell (n=7). This was a significant decrease from uptake of GAL-24 by the untreated cells (p=0.0001), but was not statistically different from the decrease caused by treatment with free β-d-galactose (p>0.05). Some PAMAM dendrimers have been recorded utilizing multiple endocytic pathways to enter highly phagocytic glia, so it remains possible that the other ~35% of the GAL-24 uptake is by these other fluid-phase endocytic pathways. Hepatocytes also have the ability to ferry particulates via lipid rafts that aid in the polarization and directionality of hepatic transport, but their utilization by GAL-24 would be unlikely due to the high specificity of the lipid rafts to signaling proteins.

Imaging of cells undergoing the same treatments as those utilized in the uptake studies confirms the hypothesis that GAL-24 is utilizing ASGP-R-mediated endocytosis. When cells are co-incubated with free β-d-galactose, localization of G2-Gal24-OH96 does not change, but the GAL-24-Cy5 signal decreases in intensity as compared to the untreated cells, what one could expect when there are additional ligands competing for binding sites. Whereas with chlorpromazine treatment, GAL-24 is mostly punctate and sequestered on the cell surface, which could indicate that the GAL-24 is binding to ASGP-R, but without the assembly of clathrin the receptor is not internalizing. In conclusion, based on these studies, GAL-24 is relatively non-toxic, binds strongly to ASGP-R, and enters hepatocytes in culture through ASGP-R-mediated endocytosis.

### Example 3: In Vivo GAL-24 Localizes Preferentially in the Liver, Specifically in Hepatocytes, of Healthy Mice

### Materials and Methods

Translational impact of this nanocarrier is dependent on its performance in vivo, achieving specific delivery to liver hepatocytes while avoiding systemic accumulation and toxicity. For this reason the pharmacokinetics of GAL-24 in healthy mice upon systemic administration were investigated. 6-8 week old C57BL6 mice were anesthetized and injected with 55 mg/kg of fluorescently labeled GAL-24 (GAL24-Cy5) via tail vein. When the mice were sacrificed at various time points, blood was sampled via cardiac puncture and the body perfused to remove background signal from free GAL24-Cy5. The first step was to determine the content of dendrimer in the liver by extracting the GAL24-Cy5 from samples of liver tissue via homogenization in a solution of 70:30 methanol (“MeOH”):DPBS that had a >90% extraction efficiency in ex vivo tissue.

### Results

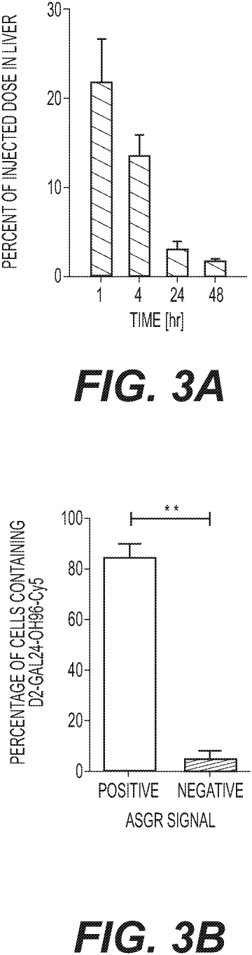

The liver extracts reveal that the GAL-24 has a high affinity for liver tissue in vivo with >20% of the injected dose (ID) in the liver 1 hr after injection and ~2%ID still in the liver at 48 hrs after injection (FIG. 3A). This uptake equates to ~250 µg dendrimer/g tissue at 1 hr and ~20 µg dendrimer/g tissue at 48 hrs. GAL-24 uptake in the liver is in line with many other liver targeting nanoparticles, which have report levels from 0.1-2% ID in the overall liver still present at 24 hours. The liver:plasma ratio for GAL-24 was 4.5 at 1 hour after injection, which increased to >20 over time as dendrimer is internalized in the liver, but is quickly removed from circulation due to its small size.

Additional mice were injected with the same dose, 55 mg/kg, of GAL-24-Cy5 and their livers were preserved for imaging. Liver sections from 1, 4, 24, and 48 hours after stained for hepatocytes with ASGP-R and sinusoidal endothelial cells (SECs) with SE-1. While hepatocytes are the major cell type of the liver, most nanoparticles that accumulate in the liver actually localize in sinusoidal endothelial cells or Kupffer cells residing in the sinusoid as they are broken down and removed from the liver instead of internalizing in hepatocytes. Confocal imaging of these liver sections shows that GAL24-Cy5 is located throughout the entire liver parenchyma with robust signal within hepatocytes. At just one hour after injection there is punctate signal overlapping with sinusoidal endothelial cells, which could be indicative of excess dendrimer being removed from the liver as equilibrium is reached as at both 4 and 24 hours after injection there is perfuse signal throughout the liver with no noticeable spots with concentrated dendrimer. Then, at 48 hours, punctate signal in SECs returns, indicating that GAL-24 is cleared from the liver via the sinusoid.

To bolster the case for the hepatocyte-targeting capabilities of GAL-24, the primary livers cells of mice injected with GAL24-Cy5 were isolated to identify hepatocytes and quantify their dendrimer content. Deeply anesthetized mice that had received a 55 mg/kg injection of GAL24-Cy5 24 hours prior were dissected prior to isolation of primary liver cells. The primary cell suspension was then stained for live cells with eFluor 450 viability dye and for hepatocytes with ASGP-R antibody. Flow cytometry analysis revealed that ~90% of the cells were alive, ~40% of which were hepatocytes. Over multiple replicates, ~85% of the live hepatocytes were positive for GAL-24 signal whereas <5% of live non-hepatocytes expressed dendrimer signal (FIG. 3B), which was a significant difference (Paired t-test, p=0.0044, n=3). Only minimal amounts of Kupffer cells were extracted with this protocol and those that were did not have Cy5 dendrimer signal. It is possible that the remaining signal is from SECs that are shuttling the excess dendrimer out of the liver. The data showed that the majority of GAL-24 is localized in liver hepatocytes.

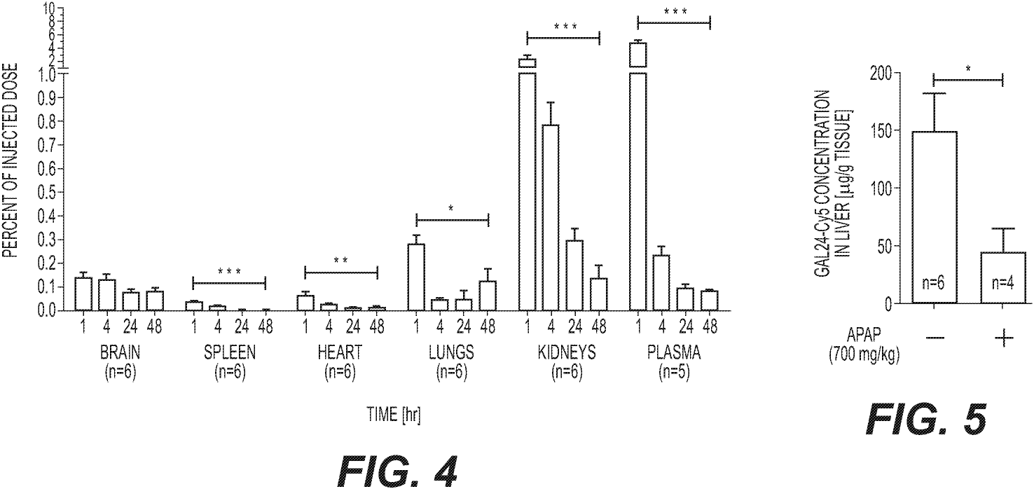

All internal organs recovered from the mice utilized in the previously described liver uptake studies were further analyzed. Upon homogenization and extraction of GAL-24 from the tissues using 70:30 MeOH:DPBS, it was revealed that, apart from the liver, Gal-24 presence was minimal throughout the body (FIG. 4). In all organs and plasma, GAL-24 concentration was highest at 1 hour post injection. There was a main effect of time and a significant decrease in dendrimer uptake between 1 and 48 hours in the spleen (F(3,20)=20.48, p<0.0001, r²=0.7544, t-test with Tukey’s correction 1 v. 48 hrp<0.0001), heart (F(3,20)=8.716, p=0.0007, r²=0.5666, t-test with Tukey’s correction 1 v. 48 hr p=0.0022), lungs (F(3,20)=9.800, p=0.0003, r²=0.5951, t-test with Tukey correction 1 v. 48 hr p=0.0235), kidneys (F(3,20)=15.67, p<0.0001, r²=0.7015, t-test with Tukey correction 1 v. 48 hr p<0.0001), and plasma (F(3,16)=87.18, p<0.0001, r²=0.9424, t-test with Tukey correction 1 v. 48 hr p<0.0001), indicating that the dendrimer is not accumulating in any off target organs and is cleared rapidly. No organ other than the liver or kidneys had greater than 1%ID of GAL-24 present at any time. The greatest remaining amount of GAL-24 at 48 hours is in the kidney, with just 0.14% ID (FIG. 4), which was anticipated as GAL-24, with a hydrodynamic diameter <5.5 nm, would likely participate in renal clearance, although at 48 hours the liver:kidney ratio of GAL-24 uptake is >12, indicating there is a strong preference for uptake in the liver despite renal clearance.

Confocal imaging of the kidney at 1 and 24 hours shows that the high dendrimer concentration there is likely due to the passing of GAL-24 through the fenestrations in the kidney glomerular capillary walls with GAL-24 signal seen colocalized with GFAP staining of the glomerulus of the kidney. Over time, dendrimer signal from the medulla vanishes, and the signal in the cortex weakens, indicating that the dendrimer is safely being cleared from the kidneys with no potentially harmful uptake. While no organ had >1 % ID of GAL-24 at 24 hours, the localization of the new dendrimer conjugates was further investigated. GAL-24 levels in the brain were comparable to those reported for hydroxyl-terminated PAMAM dendrimers in healthy brain tissue at about 0.002% ID/g tissue. Also similarly to other dendrimers, GAL-24 is seen only localized in IBA1 positive cells of the choroid plexus and is unable to cross the healthy, intact BBB 24 hours after administration. This gives further support that a small diameter and a high density of surface hydroxyl groups is critical for localization of a nanoparticle in the brain. There could be additional concerns with cardiovascular toxicities when it comes to localization of a particle in the heart, so the cardiac biodistribution of GAL-24 was also examined. Imaging shows that the minimal amount of GAL-24 present in the heart and is localized almost exclusively in cells positive for β-actin stain, indicative of cardiac endothelial cells or fibroblasts as cardiomyocytes have greatly reduced β-actin activity, minimizing the threat of negative side effects in the heart from GAL-24 administration as localization in cardiomyocytes can have incredibly damaging effects on the heart.

Finally, it was confirmed that the compound was cleared from the system in an intact form. HPLC analysis of filtered urine, feces, serum, liver, kidney, and spleen extracts revealed that the major fluorescent component throughout the body is intact GAL24-Cy5. The retention time and shape of the peak detected in the fluorescence channel from each extract at 4 and 24 hours remained as shown previously in the synthesis with no significant peak spreading or the presence of new peaks. This confirms that the design of GAL-24 is well suited for the development of stable compounds for use in vivo. Finally, paraffin embedded, sectioned, and stained kidney, liver, and spleen for Hemotoxylin and Eosin (H&E) at 48 hours and one week after injection were analyzed to determine if there were any clear signs of toxicity after a single 55 mg/kg i.v. administration of GAL-24. Kidney, liver, and spleen images from GAL-24 injected animals were indistinguishable from those taken from saline injected control animals. If the particle were not well tolerated one would expect to see immune cell infiltration, irregular cell and nuclei shape, and even cellular apoptosis. Qualitatively, the organs from GAL-24 injected animals had no obvious toxicities through one week after injection.

### Example 4: GAL-24 Maintains its Hepatocyte-Targeting Capabilities in Hepatic Necrosis

The above studies show that GAL-24 is a powerful new tool for targeting hepatocytes both in culture and from systemic injection in vivo. However, it is unlikely that a new GAL-24 based therapy would be applied to humans with fully functioning healthy livers. Therefore, the performance of GAL-24 in two models representing two of the major branches of liver diseases was evaluated: drug-induced liver failure, and diet induced liver fibrosis/cirrhosis. These two disease types have unique pathways and roles that the hepatocytes play, which means that GAL-24 may be better suited for a specific application, as there is currently only one nanoparticle on the market for treating a liver disease, and it is highly specialized to the treatment of rare hereditary autosomal transthyretin (hATTR) amyloidosis.

### Materials and Methods

A mouse model of severe acetaminophen (APAP) poisoning was used to investigate if GAL-24 maintains its highly favorable hepatocyte-targeting capabilities in the context of disease where the target cell population is dying, further strengthening its translational potential. Severe APAP poisoning was induced by intraperitoneal injection of 700 mg/kg APAP into 6-8 week old C57BL6 mice following food restriction. GAL-24 was dosed at 55 mg/kg through tail vein injection 24 hours after APAP administration. 4 hours after receiving G2-Gal24-OH96 the mice were sacrificed due to the rapid attrition observed in this model. Model establishment and hepatic necrosis was confirmed by histology and animal weight loss.

### Results

Liver tissue homogenization and extraction of the GAL-24 revealed that in this case of rampant hepatocyte oxidative stress and death, the GAL-24 was still able to localize to the liver at levels of ~45 µg/g tissue, which is about one third of what was observed in healthy animals (FIG. 5). This quantity is still well within the window of what could be utilized therapeutically, especially considering that many other disease models would not involve such high levels of hepatocyte death and the introduction of additional physical barriers such as those imposed by necrotized tissue. A segment of each diseased liver was preserved for cryopreservation and sectioning. The staining of these liver sections revealed that similarly to what is seen in healthy animals, the GAL-24 is accumulating mostly in areas positive for hepatocyte stain with an average co-localization coefficient >0.9 between dendrimer and hepatocyte signal as determined by ZEN software.

### Example 5: GAL-24 Maintains its Hepatocyte-Targeting Capabilities in Cases of Hepatic Fibrosis

### Materials and Methods

GAL-24 was further assessed in the worst-case scenario where the target population of cells is rapidly dying due to drug induced hepatic necrosis such as in diseases of hepatic dysfunction and fibrosis. For this study, a rat model of non-alcoholic steatohepatitis (NASH) induced by high-fat methionine-choline-deficient (HF-MCD) diet was employed. In NASH, hepatic stellate cells become activated and induce fibrosis of the liver tissue as a response to the build-up of fatty acids. Recent therapeutic developments have focused on treating stellate cells. However, hepatocytes still play a role in both the initial processing of fatty acids built up in the liver, and the inflammatory response to fibrosis, which could be therapeutic targets for GAL-24. To mimic this early stage of NASH with fatty acid build up before the development of fibrosis, SD rats were fed a HF-MCD diet for only six weeks before 20 mg/kg dendrimer injection.

### Results

Rats were sacrificed 24 hours after injection, and robust uptake of GAL24-Cy5 in the liver tissue was observed. Based on morphology and co-localization, it was clear that the dendrimer remains internalized in hepatocytes in rats as well as mice, and not Kupffer cells as stained for with lectin, which are also activated in NASH. One liver was homogenized for dendrimer uptake. This singular liver had a dendrimer content of 2% ID/g tissue, which is much higher than the 24-hour uptake in mouse livers observed previously.

### Example 6: Synthesis of Galactosylated Generation 4 Hydroxyl-Terminated PAMAM Dendrimer

### Materials and Methods

To investigate the effect of galactosylation on dendrimer binding and transport kinetics, galactose was appended to the surface of D4-OH (D4-Gal) followed by the additional attachment of Cy5 fluorophore (Gal-D4-Cy5) to make the dendrimer observable in vitro and in vivo. The monofunctional D4-OH was modified to be trifunctional for the attachment of both galactose and Cy5 while maintaining majority of the surface hydroxyl groups unmodified as it has been shown that the density and presence of the surface hydroxyl groups is key to the favorable biodistribution and toxicity profile of D4-OH. The synthesis of Gal-D4-Cy5 closely mimics the previously published synthesis of Mannose-D4-Cy5 (Sharma A et al., J Control Release. 2018 Aug 10; 283: 175-189).

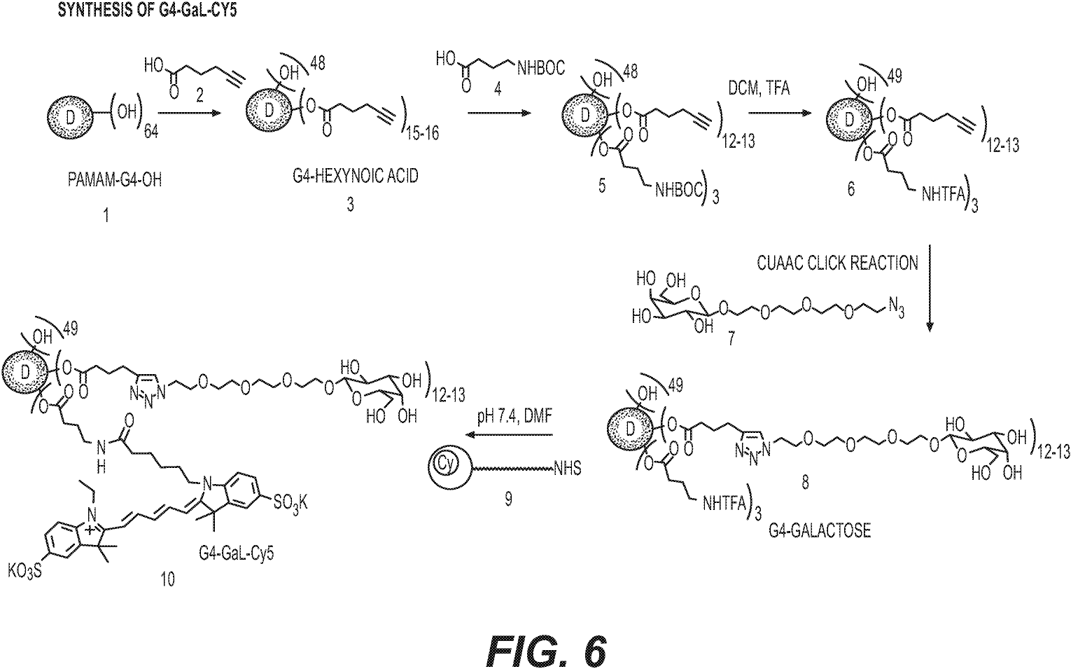

The synthesis of Gal-D4-Cy5 involved two major steps: 1) preparation of trifunctional dendrimer with alkyne and amine surface groups, and 2) conjugation of β-galactose linker and Cy5 to the trifunctional dendrimer (FIG. 6).

Trifunctional dendrimer was synthesized by EDC/DMAP esterification reaction with D4-OH (1) and hexynoic acid (2) to form D4-Hexyne (3) with 12 hexyne linkers attached and 52 hydroxyl groups remaining unmodified. Next, D4-hexyne was reacted with GABA-BOC-OH (4) via EDC/DMAP coupling reaction to form protected trifunctional dendrimer (5). Finally, the BOC group was deprotected with TFA to make the final trifunctional dendrimer (6) having 12 alkyne and 3 amine terminal groups. Galactose and Cy5 were then conjugated stepwise to the dendrimer surface, with the addition of galactose-TEG-N₃ (7) occurring first through CuAAC reaction to form compound (8) and finally Cy5-NHS ester (9) was conjugated to the dendrimer surface using activated acid-amine coupling reaction to yield Gal-D4-Cy5 (10). Non-fluorescent D4-Gal was synthesized simply through a CuAAC reaction between intermediates (3) and (7). All products and intermediates were purified through dialysis, combiflash, or HPLC and characterized with 1H-NMR and HPLC.

### Results

Addition of galactose to the dendrimer surface did not significantly increase the diameter of the dendrimer or the zeta potential, yielding a neutral conjugate with a diameter of ~5 nm.

### Example 7: D4-Gal Binding Affinity and Hepatocyte Uptake in Vitro

### Materials and Methods

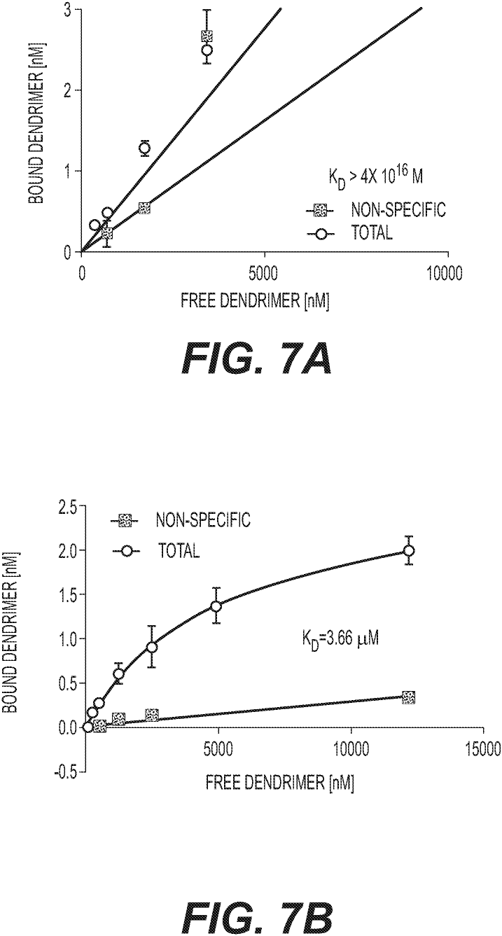

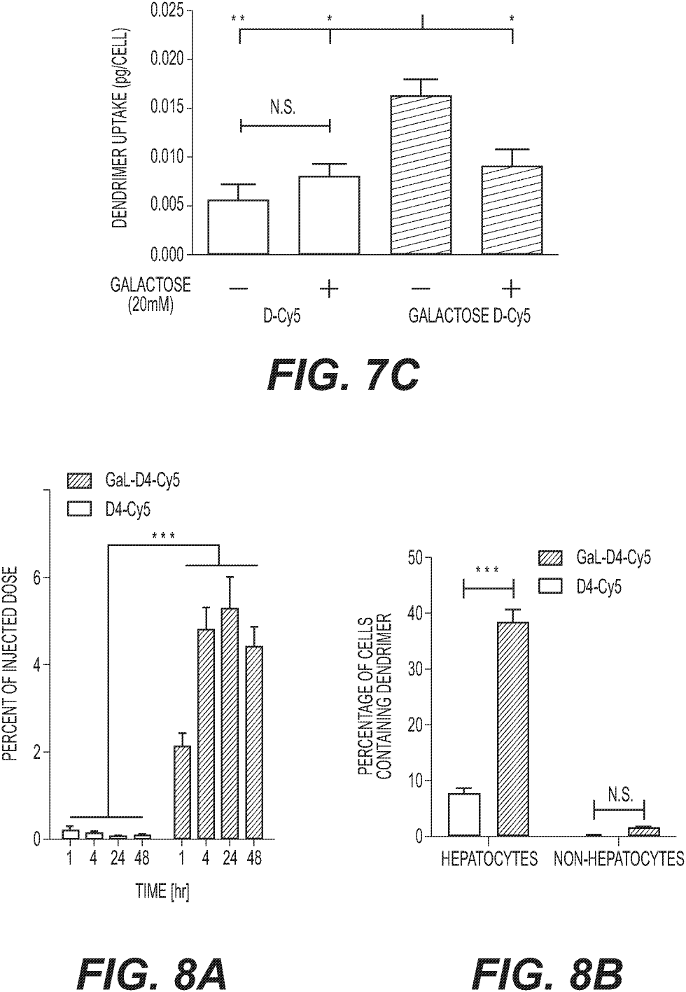

The major design criterion for D4-Gal was multivalent binding to ASGPR for internalization in hepatocytes. Binding affinity for ASGPR was determined for D4-Gal. A cell surface binding assay on HEPG2 cells was utilized, which express on the order of 100,000 ASGPR per cell, and to which the D4-Gal was non-toxic up to 1000 µg/mL as determined by MTT assay (FIG. 7A). This method was chosen as it has been reported that binding affinity to ASGPR can change over 100-fold between free receptor systems, like surface plasmon resonance and cell based assays. Based on the total binding isotherm and the correlated non-specific binding quantification through the introduction of ASGPR blocking peptide, the binding affinity was determined: K_{D} of D4-Gal to ASGPR was 3.66 µM (FIG. 7B, r2=0.9192 total, r2=0.8297 non-specific, 95%CI: 2.025 < KD < 6.562 µM, n=3). D4-OH had very low binding to ASGPR that it could not be accurately measured with the GraphPad prism program as total binding was almost equal to non-specific binding, indicating no preferential binding to ASGPR.

K_{D} of free galactose is estimated to be between 2 and 5 mM, making the binding of D4-Gal to ASGPR about 1000-fold stronger than free galactose sugar. The observed improvement in binding affinity is most likely due to the impact of both multivalency and the cluster glycoside effect. While galactose has a strong affinity for ASGPR, a common ligand with improved binding affinity is N-acetylgalactosamine (GalNAc), which has a micromolar scale monovalent binding affinity. GalNAc conjugates are widely used, but some have reported hepatotoxicities as well as off-target binding. Accordingly, galactose was chosen for its increased capacity for clinical translation. Additionally, the experimental binding affinity of D4-Gal was in a similar range to those of multivalent glycopolymers of GalNAc, which were found to be in the range of 7 to 0.3 µM, although this is surpassed by the Alnylam Pharmaceuticals trivalent glycocluster system, which has a 2 nM binding affinity to ASGPR.

### Results

The impact of galactosylation on dendrimer uptake by HEPG2 cells in culture was investigated. Following 24 hours of treatment with medium containing either Gal-D4-Cy5 or D4-Cy5, HEPG2 cells were found to have a significantly increased uptake of Gal-D4-Cy5 at 0.016 pg dendrimer/cell as opposed to unmodified D4-Cy5 which was only present at levels of 0.006 pg/cell (p=0.007, FIG. 10C, two-way ANOVA for dendrimer and treatment F(1,12)=10.43, p=0.0072, n=4). HEPG2 cells as also pretreated and co-incubated with free galactose sugar to determine if increased uptake was in fact due to enhanced binding to ASGPR, and it was observed that galactose treatment reduced Gal-D4-Cy5 uptake to 0.0097, significantly lower than untreated Gal-D4-Cy5 uptake (p=0.0223), while having no impact on D4-Cy5 uptake. Based on the results of the binding study, the presence of 20,000-fold more free ligand than dendrimer would be sufficient to quench ASGPR mediated uptake of Gal-D4-Cy5, which was observed as co-incubation with galactose resulted in uptake of Gal-D4-Cy5 equivalent to that of D4-Cy5. Overall uptake of both dendrimers is much lower than that observed previously in macrophages, but can be attributed to the fact that unlike active microglia and macrophages, hepatocytes are not actively phagocytosing.

Non-zero, similar uptake of free galactose-inhibited Gal-D4-Cy5 and D4-Cy5 indicates that the modified dendrimer still maintains its ability to enter cells through non-specific fluid phase endocytosis, which will have interesting implications on the development of future targeted and drug-loaded D4-OH dendrimer conjugates. Previous studies with galactose dendrimers have shown that both co-incubation with galactose as well as treatment with chlorpromazine, which blocks clathrin-mediated endocytosis, like with ASGPR, diminish dendrimer uptake in similar amounts, giving additional credence to the assertion that the increased HEPG2 uptake observed here is due to ASGPR binding and internalization. The dendrimer uptake was additionally observed through confocal microscopy (FIG. 10D), where it was evident that Gal-D4-Cy5 signal was much more prevalent than when co-treated with galactose as well as D4-Cy5 in any condition. Gal-D4-Cy5 signal is also perfuse throughout the cytoplasm, an indicator that the dendrimer escapes from endosomes once internalized in the cell, possibly via the proton sponge effect, as nanoparticles entrapped in vesicles would appear punctate in images.

### Example 8: Pharmacokinetics of Intravenous Gal-D4-Cy5 in Vivo in Healthy Mice

### Materials and Methods

The D4-Gal conjugate maintains its hepatocellular targeting in vivo as D4-OH is known to clear rapidly through the kidneys due to renal clearance as its diameter is ~4 nm, which is minimally increased to ~5 nm with the addition of galactose to the surface. For this study, healthy C57BL6 mice were used and administered 55 mg/kg of D4-Cy5 or Gal-D4-Cy5 via tail vein injection. Biodistribution was assessed at 1, 4, 24, and 48 hours post injection. The choice of healthy C57BL6 mice was to aid in future studies on acetaminophen poisoning as the C57BL6 mouse is the superior model choice due to their similar metabolism of acetaminophen by cytochrome p450, while observing the interaction of the dendrimer with fully functional tissue.

Mouse liver tissue was homogenized in methanol to extract the dendrimer, which revealed enormous presence of Gal-D4-Cy5 in the liver. Gal-D4-Cy5 uptake in liver is significantly greater than that of D4-Cy5 at all times (FIG. 8A), two-way ANOVA, F(1,34)=274.8, p<0.0001, n=4-6), with a ~90-fold difference between Gal-D4-Cy5 and D4-Cy5 at the 24 hour peak of uptake. Localization of Gal-D4-Cy5 in the liver is maintained between 24 hours and 48 hours at ~5% of the total injected dose (24 v 48 hour Gal-D4-Cy5, Tukey’s multiple comparison t-test, p=0.6864). This sustained delivery to the liver is surprising with many liver-targeting nanoparticles clearing more rapidly, or never achieving as great of uptake in the liver (He, H., et al., Biomaterials, 2017. 130: p. 1-13; Zou, Y., et al., Journal of Controlled Release, 2014. 193: p. 154-161; Tsend-Ayush, A., et al., Nanotechnology, 2017. 28(19): p. 195602). IVIS imaging of ex vivo livers enabled visualization of the stark difference between D4-Cy5 and Gal-D4-Cy5 accumulation and confirmed that Gal-D4-Cy5 localized homogeneously throughout the liver.

### Results

Future therapies for liver diseases and/or disorders such as drug-induced liver failure, hepatocellular carcinoma, and hepatitis, need to localize in hepatocytes or have hepatocyte-specific mechanisms of action, so the cellular localization of Gal-D4-Cy5 within the liver through was further analyzed using both confocal imaging and flow cytometry of primary mouse livers. Imaging of Gal-D4-Cy5 in the liver as seen through tissue sections stained to identify both hepatocytes and sinusoidal endothelial cells shows that Gal-D4-Cy5 signal almost exclusively colocalizes with hepatocyte signal. This is in stark contrast to D4-Cy5, which was not present in visible quantities in the liver, as well as other nanoparticles that accumulate in the liver and only localize in sinusoid as they are removed from systemic circulation through the mononuclear phagocyte system. Gal-D4-Cy5 signal was still readily observable within hepatocytes at a week after injection, which is uniquely suited for the delivery of drugs which need a longer release window. Confocal imaging is not sufficient to prove targeted uptake in hepatocytes, so flow cytometry was used to analyze liver cells that were isolated from mice injected with either D4-Cy5 or Gal-D4-Cy5 and then labeled for hepatocytes with ASGPR antibody (FIG. 8B). About 40% of all live hepatocytes were positive for Gal-D4-Cy5 24 hours after injection, which is greater than the proven hepatocyte-specific uptake for many currently available nanosytems. This uptake is significantly greater than Gal-D4-Cy5 in cells that were not hepatocytes (two-way ANOVA F(1,12)=604.5, p<0.0001, n=3, Tukey’s multiple comparison t-test p<0.0001), or D4-Cy5 in either hepatocytes or non-hepatocytes (p<0.0001 in both cases),. This data conclusively shows that systemically injected D4-Gal specifically targets hepatocytes, maintaining localization for up to one week following injection.

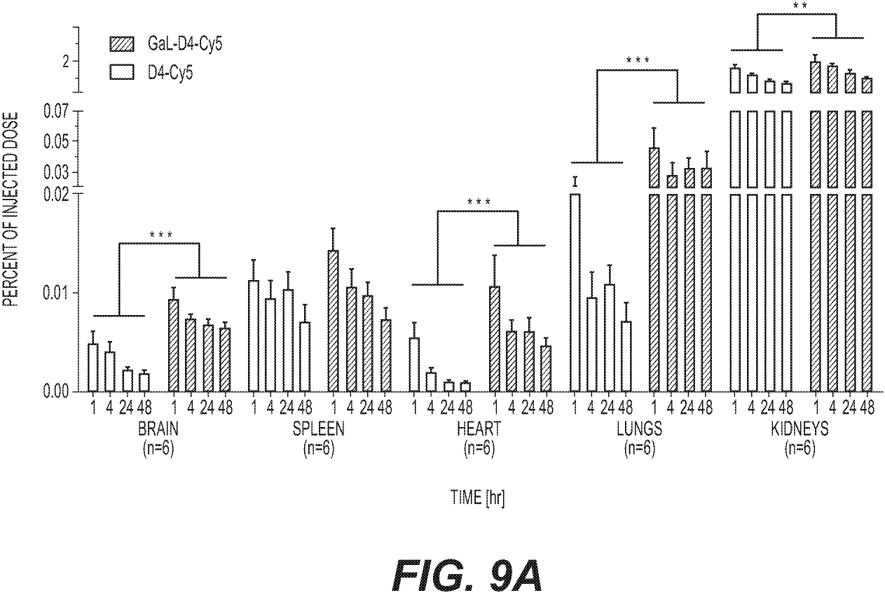

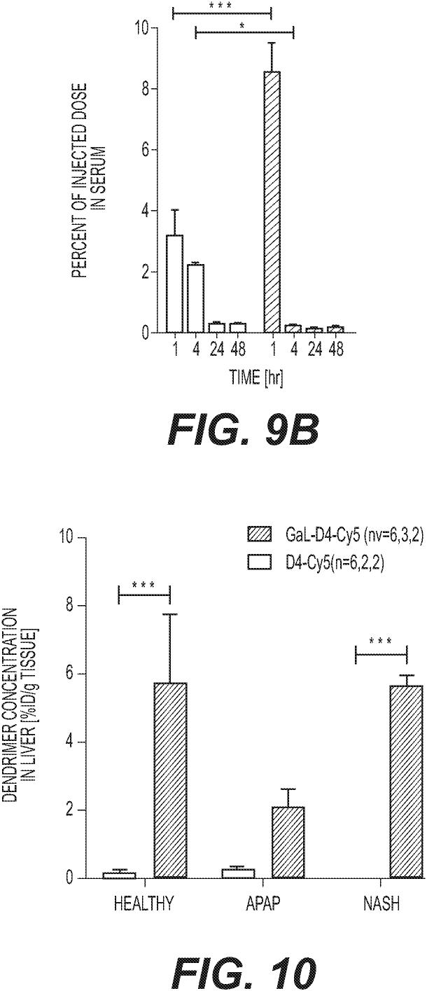

The localization of Gal-D4-Cy5 could not be considered highly specific targeting until it is shown that there is not significant uptake in off-target tissue, so the full body pharmacokinetics of Gal-D4-Cy5 and D4-Cy5 in mice was further analyzed (FIGS. 9A and 9B). Two-way ANOVA analysis indicated that Gal-D4-Cy5 was present in significantly higher quantities than D4-Cy5 in the brain (F(1,40)=73.57, p<0.0001), heart (F(1,38)=28.47, p<0.0001), lungs (F(1,40)=20.53, p<0.0001), kidneys (F(1,40)=10.48, p=0.0024), and plasma (F(1,24)=3.994, p=0.0571); however, in none of these organs was there a main effect of the interaction between dendrimer and time (p=0.4580, 0.9093, 0.9433, 0.9516 respectively) indicating that while there is increased uptake, the accumulation and clearance rates of Gal-D4-Cy5 from these organs is not significantly different from that of D4-Cy5. The increased uptake levels may be due to the presence of sugar receptors such as sodium-glucose cotransporter type 1 and glucose transporter 1, that are expressed throughout the body and have slight affinities for galactose (Coady, M.J. et al., American Journal of Physiology-Renal Physiology, 2017. 313(2): p. F467-F474; Mueckler, M. and B. Thorens, Molecular aspects of medicine, 2013. 34(2-3): p. 121-138). However, any increase in uptake is negligible since no organs except kidneys contain more than 0.05% of the total injected dose at 48 hours and the kidneys contain less than 1% ID, which is similar to that of D4-Cy5 (Tukey’s multiple comparison t-test, p=0.9420). There was a main effect of interaction in plasma (F(3,24)=18.57, p<0.0001), which was most likely due to the drop off of Gal-D4-Cy5 concentration in serum, which may have arisen from the rapid accumulation of Gal-D4-Cy5 in the liver, as opposed to D4-Cy5 that is gradually filtered out by the kidney proximal tubules over 24 hours.

The high specificity of Gal-D4-Cy5 to liver hepatocytes and no other organs or cells of the body to a significant degree makes it a desirable candidate for drug delivery to hepatocytes. Next, the ability of Gal-D4-Cy5 to maintain its liver targeting capability in the context of liver disease was investigated. Liver uptake of both Gal-D4-Cy5 and D4-Cy5 was assessed in a rat model of high-fat methionine-choline deficient (HF-MCD) diet induced non-alcoholic steatohepatitis (NASH) and a mouse model of acetaminophen (APAP) induced liver failure. In both models Gal-D4-Cy5 continued to outperform D4-Cy5 with 5.66%ID/g tissue localizing in the NASH liver and 2.06%ID/g tissue in the APAP overdose model, which is compared to <0.3%ID/g tissue of D4-Cy5 being taken up by the liver in any model (FIG. 10). There was a main effect of disease and dendrimer (two-way ANOVA, F(2,15)=4.717, p=0.0257), and the uptake of Gal-D4-Cy5 was significantly greater than that of D4-Cy5 in the NASH model (Sidak’s multiple comparison t-test, p=0.0007, n=2), which was clearly observed in confocal imaging of rat liver sections. When images were analyzed under high magnification it was also apparent that the Gal-D4-Cy5 had maintained the same hepatocyte-specific localization as observed in the healthy mouse tissue previously. Gal-D4-Cy5 uptake in the APAP overdose model was reduced ~3-fold from healthy tissue as opposed to the NASH model, which had almost equivalent uptake as compared to healthy mice. This discrepancy is most likely due to the fact that the NASH model involves highly functioning live hepatocytes as the disease has not yet progressed to fibrosis and cirrhosis, whereas just 24 hours after APAP administration there is rampant tissue necrosis and hepatocyte death in the APAP overdose model, reducing the number of live hepatocytes for Gal-D4-Cy5 to target.

### Example 9: Synthesis of Gal-D4-NAC

Conjugation of N-acetyl cysteine (NAC) to the surface of D4-Gal (Gal-D4-NAC) was achieved through a glutathione-sensitive disulfide linker to allow release once internalized in the hepatocytes but remain stable in solution and in plasma. Systemic NAC therapy is already the standard-of-care for the clinical presentation of APAP poisoning, but becomes ineffective at later time points or increasingly large doses as is shown by the treatment nomogram. It was hypothesized that if NAC could be delivered more rapidly and directly to the hepatocytes that need it, then the treatment window for severe APAP poisoning could increase, reducing mortality and the need for liver transplants.

A NAC-loaded D4-Gal conjugate was synthesized (FIG. 11) with 10 molecules of galactose on the surface and 15 molecules of NAC connected to the dendrimer with a cleavable, glutathione sensitive linker. PAMAM D4-OH (11) was modified with 25 hexynoic acid groups through EDC/DMAP coupling esterification to create a bifunctional dendrimer, D4-hexyne (12). An azido-PEG-4-amine linker (13) for eventual NAC conjugation was then attached to roughly 60% of the hexyne groups through a copper catalyzed CLICK reaction, resulting in hexyne-D4-PEG-NH₂. The remaining hexyne groups were then reacted with β-galactose-TEG-azide (7, FIG. 6) to create Gal-D4-PEG-NH2 (14), which was further reacted with NAC-SPDP-NHS (15) ester through an amide-ester reaction to yield the final product, Gal-D4-NAC (16) having 10 galactose and 15 NAC on the surface. Final products and intermediates were purified and thoroughly characterized with HPLC and ¹H-NMR. The size and zeta potential of D4-NAC did not diverge greatly from those of D4-OH in previous studies, indicating that the addition of NAC will likely not have a major effect on the previously analyzed biodistribution of D4-Gal as it has been previously shown that D4-Cy5 and NAC-D4-Cy5 have similar localization in vivo.

### Example 10: Systemic D4-Gal Mediated N-acetyl Cysteine Treatment in a Mouse Model of Severe Acetaminophen Poisoning

### Materials and Methods

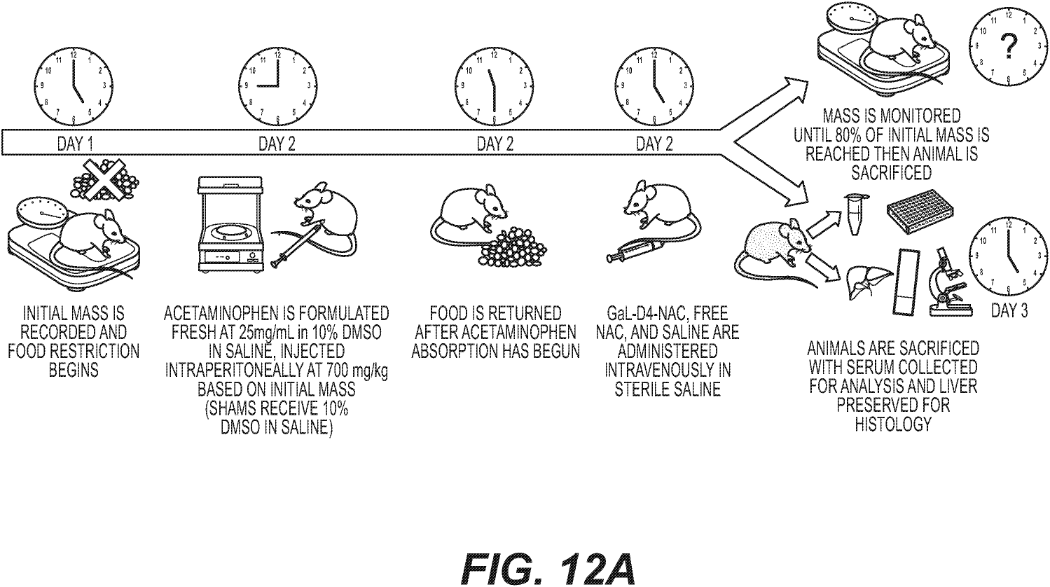

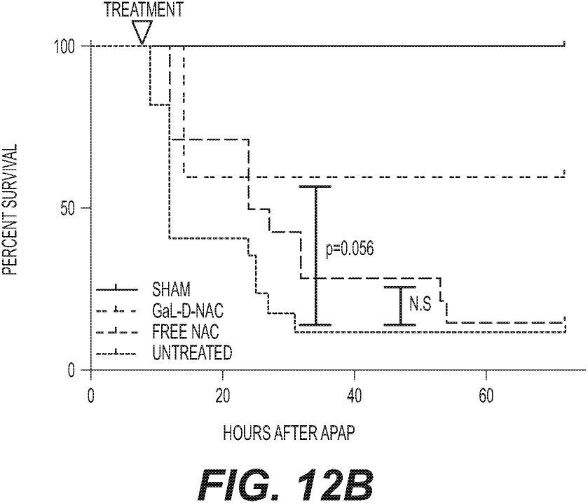

A model of severe acetaminophen poisoning was established through the administration of 700 mg/kg APAP i.p. to C57BL6 mice (FIG. 12A). There was a high mortality rate, with >90% of mice given APAP overdose dying with 72 hours regardless of treatment with free NAC (FIG. 12B). FIG. 12B represents the survival of healthy sham (black) and saline (red), free NAC (green), and Gal-D4-NAC (blue) treated animals as determined by mass reduction below 80% of the initial animal mass. This model replicates the effects of APAP overdose that result in fulminant liver failure and are considered untreatable with the current clinical NAC regiment. Additionally, to mimic delayed treatment due to late arrival at the hospital, animals eight hours after APAP overdose were also treated as opposed to the pre- and co-treatments with nanoparticles that are typically reported in this model. Both dendrimer and free drug treated animals received 100 mg/kg of NAC, which is <10% of the dose that would be administered over 24 hours by i.v. infusion in the clinic.

Next liver necrosis and function were assessed through analysis of images from liver sections isolated at time of death and stained for hematoxylin and eosin. Healthy liver shows an organized structure of hepatocytes with clear sinusoidal spaces and a uniform pink color without accumulation of debris. After receiving an overdose of APAP, liver structure is essentially destroyed. There is lack of visible sinusoidal space, heterogeneity in nuclear shape and size, loss of hepatocyte borders and organization, accumulation of protein adducts, and hepatocellular vacuolation indicative of a liver with minimal to no function.

### Results

Just a single dose of Gal-D4-NAC at 100 mg/kg on a NAC basis dramatically preserved liver architecture with clear hepatocyte integrity and organization. There is some clogging of the sinusoidal space by debris and hepatocellular vacuolation, but neither is not as dramatic as the untreated animals. Taken together these two assessments show that Gal-D4-NAC can save previously unsalvageable livers in both form and function, making D4-Gal a powerful tool for accessing and treating hepatocytes.

We claim:

1. A method for treating or preventing one or more symptoms of a liver disease and/or disorder in a subject in need thereof, comprising
administering to the subject a formulation comprising galactosylated dendrimers complexed to, covalently conjugated to, or having intra-molecularly dispersed or encapsulated therein one or more therapeutic or prophylactic agents,
wherein the formulation is administered in an amount effective to treat, alleviate or prevent one or more symptoms of the liver disease and/or disorder.

2. The method of claim 1, wherein the one or more liver disease and/or disorder is selected from the group consisting of inflammatory liver diseases, non-alcoholic steatohepatitis, drug-induced liver failure, hepatitis, liver fibrosis, liver cirrhosis, hepatocellular carcinoma, and combinations thereof.

3. The method of claim 1, wherein the galactosylated dendrimers are hydroxyl-terminated dendrimers.

4. The method of claim 1, the galactosylated dendrimers are generation 4, generation 5, generation 6, generation 7, or generation 8 poly(amidoamine) dendrimers.

5. The method of claim 1, wherein the galactosylated dendrimers are made of galactose and oligoethylene glycol building blocks.

6. The method of claim 5, wherein the galactosylated dendrimers are made of galactose and oligoethylene glycol building blocks, of generation 1 with 24 hydroxyl terminal groups, generation 2 with 96 hydroxyl terminal groups, generation 3 with 384 hydroxyl terminal groups, or generation 4 with 1536 hydroxyl terminal groups.

7. The method of claim 5, wherein the galactosylated dendrimers are a generation 2 dendrimer where 24 galactose units comprise the outer layer and six galactose units are embedded in the dendrimer backbone connected through tetraethylene glycol units.

8. The method of claim 1, wherein the therapeutic agent is an agent selected from the group consisting of non-steroidal anti-inflammatory agents, corticosteroid anti-inflammatory agents, gold compound anti-inflammatory agents, immunosuppressive, and anti-oxidant agents.

9. The method of claim 1, wherein the anti-inflammatory agent is N-acetyl cysteine.

10. The method of claim 1, wherein the therapeutic agent is vitamin E.

11. The method of claim 1, wherein the formulation is administered in an amount effective to reduce one or more serum levels of alanine aminotransferase (ALT), aspartate aminotransferase (AST), triglyceride (TG), gamma-glutamyltrasferase (GGT), total cholesterol (TC), low density lipoprotein (LDP), fasting blood sugar, or combinations thereof.

12. The method of claim 1, wherein the formulation is administered in an amount effective to reduce one or more of steatosis, inflammation, ballooning, fibrosis, cirrhosis, or combinations thereof.

13. The method of claim 1, wherein the formulation is administered in an amount effective to reduce lobular inflammation in the liver.

14. The method of claim 1, wherein the formulation is administered in an amount effective to reduce the amount or presence of one or more pro-inflammatory cells, chemokines, and/or cytokines in the liver.

15. The method of claim 14, wherein the formulation is administered in an amount effective to reduce one or more pro-inflammatory cytokines.

16. The method of claim 1 wherein the formulation is formulated for intravenous or intraperitoneal administration.

17. The method of claim 1, wherein the formulation is formulated for oral administration.

18. The method of claim 1, wherein the formulation is administered via the intravenous or intraperitoneal route.

19. The method of claim 1, wherein the formulation is administered via oral administration.

20. The method of claim 1, wherein the formulation is administered prior to, in conjunction, subsequent to, or in alternation with treatment with one or more additional therapies or procedures.

21. The method of claim 20, wherein the one or more additional procedures include administering one or more therapeutic, prophylactic and/or diagnostic agents to prevent or treat one or more symptoms of associated diseases or conditions of liver injuries such as infections, sepsis, diabetic complications, hypertension, obesity, high blood pressure, heart failure, kidney diseases, and cancers.

22. A pharmaceutical formulation for use in the method of claim 1.

23. A method of making dendrimers with a plurality of surface galactose comprising
(a) preparing a hypercore by performing propargylation of a first monomer, wherein the first monomer comprises two or more reactive groups for propargylation;
(b) conjugating one azide group onto glycosidic linkage at C1 of galactose via a linker to generate a galactose hyper monomer;
(c) mixing the hypercore and the galactose hyper monomer for copper (I) catalyzed alkyne azide click chemistry to yield a generation 1 dendrimer;
(d) conjugating allyl groups on four of reactive groups of the galactose hyper monomer on the generation 1 dendrimer;
(e) mixing the generation 1 dendrimer with galactose that is conjugated with an azide group for copper (I) catalyzed alkyne azide click chemistry to yield generation 2 dendrimers.

24. The method of claim 23, wherein the hypercore in step (a) is a hexapropargylated core.

25. The method of claim 23, wherein conjugating one azide group onto glycosidic linkage at C1 of galactose in step (b) is via a polyethylene glycol linker.

26. The method of claim 23, wherein the galactose that is conjugated with an azide group in step (e) is β-Gal-PEG4-azide.

27. The method of claim 23, wherein the generation 2 dendrimer from step (e) is a dendrimer with 24 galactose units forming an outer layer and six galactose units embedded within the dendrimer backbone connected through tetraethylene glycol units.

28. The method of claim 23, wherein the dendrimer is further complexed and/or conjugated to one or more therapeutic, prophylactic, and/or diagnostic agents.

29. A galactosylated dendrimer comprising galactose and oligoethylene glycol building blocks.

30. The galactosylated dendrimer of claim 29, wherein the galactosylated dendrimers are made of galactose and oligoethylene glycol building blocks, of generation 1 with 24 hydroxyl terminal groups, generation 2 with 96 hydroxyl terminal groups, generation 3 with 384 hydroxyl terminal groups, or generation 4 with 1536 hydroxyl terminal groups.

31. The galactosylated dendrimer of claim 29, wherein the galactosylated dendrimers are a generation 2 dendrimer where 24 galactose units comprise the outer layer and six galactose units are embedded in the dendrimer backbone connected through tetraethylene glycol units.

32. The galactosylated dendrimer of claim 29, wherein the galactosylated dendrimer further comprises one or more therapeutic, diagnostic, or prophylactic agents.

33. The galactosylated dendrimer of claim 29, wherein the therapeutic or prophylactic agent is N-acetyl cysteine.
